(19) United States
(12) Patent Application Publication
(10) Pub. No.: US 2024/0091356 A1
(43) Pub. Date: Mar. 21, 2024

(54) BI-SPECIFIC CAR T CCELLS FOR B CELL MALIGNANCIES

(71) Applicant: H. Lee Moffitt Cancer Center and Research Institute, Inc., Tampa, FL (US)

(72) Inventor: Marco Davila, Tampa, FL (US)

(21) Appl. No.: 18/262,287

(22) PCT Filed: Jan. 14, 2022
(86) PCT No.: PCT/US2022/070199

§ 371 Date: Jul. 20, 2023

Related U.S. Application Data
(60) Provisional application No. 63/142,186, filed on Jan. 27, 2021.

(51) Int. Cl. A61K 39/00; A61P 35/02; C07K 14/705; C07K 14/725; C07K 16/28

(52) U.S. Cl. CPC A61K 39/4631; A61K 39/4611; A61K 39/464412; A61K 39/464424; A61P 35/02; C07K 14/7051; C07K 14/70517; C07K 14/70521; C07K 16/2803; C07K 16/2887; A61K 2239/29; A61K 2239/48; C07K 2317/31; C07K 2317/622; C07K 2319/02; C07K 2319/03

(57) ABSTRACT

Disclosed are compositions and methods for targeted treatment of myeloid and B cell malignancies. In particular, chimeric antigen receptor (CAR) T cells are disclosed that can be used with adoptive cell transfer to target and kill myeloid and B cell malignancies with reduced antigen escape. Therefore, also disclosed are methods of providing an anti-tumor immunity in a subject with a myeloid and B cell malignancies that involves adoptive transfer of the disclosed CAR T cells.

### CROSS-REFERENCE TO RELATED APPLICATIONS

This application claims benefit of U.S. Provisional Application No. 63/142,186, filed Jan. 27, 2021, which is hereby incorporated herein by reference in its entirety.

### SEQUENCE LISTING

This application contains a sequence listing filed in electronic form as an ASCII.txt file entitled “320103_2110_Sequence_Listing_ST25” created on Dec. 7, 2021, and having 11,286 bytes. The content of the sequence listing is incorporated herein in its entirety.

### BACKGROUND

A major advance for T cell therapy was the chimeric antigen receptor (CAR), which is a single chain variable fragment (scFv) derived from an antibody fused to signaling domains from a T cell receptor (TCR). CAR designs that include a co-stimulatory domain, such as CD28 or 41BB, enhance in vivo CAR T cell function. The therapeutic promise of CAR T cells was realized when complete remission (CR) vrates of 90% were reported after treating B cell acute lymphoblastic leukemia (B-ALL) with CD19-targeted CAR T cells. In fact, there are 3 new FDA-approved indications for CD19-targeted CAR T cells. However, with increasing numbers of patients treated, challenges have become evident, such as high relapse rates for B-ALL and/or low response rates for Diffuse Large B cell Lymphoma (DLBCL). Some of these poor outcomes may be attributed to CAR design, for example CD28 is associated with T cell exhaustion, and/or CAR production since the outcome of patients treated with 41BB-based CAR T cells correlate with T cell quality, which is associated with memory T cell phenotypes. Furthermore, despite the success of CAR T cells for B cell malignancies it remains to be seen if these outcomes will be translated to other malignancies.

### SUMMARY

The disclosed compositions and methods are based on efforts to rationally optimize co-stimulation to reduce CAR T cell exhaustion and enhance persistence, to develop an AAPC system that enhances production of enriched memory CAR T cells, and to develop mono- and multi-antigen targeted CARs for myeloid malignancies and B cell malignancies to prevent antigen escape.

Bi-specific CAR polypeptides are disclosed that can be used with adoptive cell transfer to target and kill B cell malignancies with reduced antigen escape. The disclosed bi-specific CAR polypeptides contain in an ectodomain an anti-CD19 binding agent and an anti-CD20 binding agent that can bind CD19- and CD20-expressing cancer cells, respectively. Also disclosed is an immune effector cell genetically modified to express the disclosed CAR polypeptide.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

Also disclosed are isolated nucleic acid sequences encoding the disclosed CAR polypeptides, vectors comprising these isolated nucleic acids, cells containing these vectors, and cells comprising one or more of the herein described CAR polypeptides.

In some embodiments, the disclosed CAR T cell exhibits an anti-tumor immunity when the antigen binding domain of a CAR polypeptides binds to CD19 and CD20.

Also disclosed is a method of providing an anti-tumor immunity in a subject with a myeloid or B cell malignancies that involves administering to the subject an effective amount of a CAR T cell disclosed herein. In some cases, the myeloid or B cell malignancies comprises Acute Myeloid Leukemia (AML), blastic plasmocytoid dendritic cell neoplasm, hairy cell leukemia, or Acute Lymphoblastic Leukemia.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B contain bar graphs showing CAR candidates activate NFKB. NFKB reporter cells were transduced with γ-retrovirus containing CD19 (FIG. 1A) or CD22 (FIG. 1B) CARs. Cell lysates from transduced or untransduced cells were used for luciferase assay. Bioluminescence indicates the level of NFKB activation. h19BBzGFP, positive control; 1A10 etc., hybridoma cell IDs from which CAR scFvs were derived.

FIG. 2 is a bar graph showing HL CAR expression. CD19-, CD20- or CD22-targeted CAR T cells were produced and CAR expression was analyzed by flow cytometry. GFP % reflects CAR expression. HL, heavy chain in front of light chain orientation for scFv design.

FIG. 3 contains graphs showing CTL with HL CARs. CD19-, CD20- or CD22-targeted CAR T cells were produced and co-cultured with CD19-, CD20- or CD22-expressing target cells at a 10:1 ET ratio. Target cell killing was monitored on an xCELLigence RTCA system. 0:1. Normalized cell index reflects cell growth. UT, untransduced T cell.

FIG. 4 contains bar graphs showing 24-hour cytokine expression with CD19 HL CAR.

FIG. 5 contains bar graphs showing 24-hour cytokine expression with CD20 HL CAR.

FIG. 6 contains bar graphs showing 24-hour cytokine expression with CD22 HL CAR.

FIG. 7 is an illustration of an example embodiment for a bispecific CAR polypeptide disclosed herein.

FIGS. 8A to 8E show multiple co-stimulatory domains enhance chimeric antigen receptor (CAR) T cell memory phenotype. FIG. 8A is a schematic of CAR constructs. All include 5′ long terminal repeat (LTR), CD8 signal peptide (black bar), single-chain variable fragment with a variable heavy chain connected with glycine/serine linker to a variable light chain (VH-G/S-VL), CD8α transmembrane and hinge domain, co-stimulatory, and CD3ζ endodomain, and 3′ LTR. FIGS. 8B-8D show CAR T cells stimulated with irradiated 3T3-hCD19 target cells at a 10:1 E:T ratio for 24 hours. Cells were then collected, and phenotype determined by flow cytometry. FIG. 8B shows surface CAR expression on live CD3+CAR+ cells measured by median fluorescent intensity within protein L+ cells. FIG. 8C shows CD4/CD8 ratio on live CD3+CAR+ T cells. FIG. 8D shows central memory phenotype (CCR7⁺CD45RA⁺) among CD3+CAR+cells at 24 hours. FIG. 8E shows central memory phenotype (CCR7+CD45RA+) among CD3⁺CAR⁺ cells at weeks 1, 2 and 3 after repeated weekly antigen stimulation with CD19⁺ target cells. Data are shown from two healthy donors (HD). Data are shown as mean±SD. One-way analysis of variance (ANOVA) was performed with Dunnet's multiple comparison test against h19BB06z for FIGS. 8B-8D. Two-way ANOVA was performed with Dunnet's multiple comparison test against h19BB06z for (E).**p<0.01, ***p<0.001, ****p<0.0001.

FIGS. 9A to 9E show increased phospho-lymphocyte-specific protein tyrosine kinase (pLCK) enhances in vitro function of h19BB06z chimeric antigen receptor (CAR) T cells. FIGS. 9Ai to 9Aiv show CAR T cells were stimulated with irradiated 3T3-hCD19 cells at a 10:1 E:T ratio. After 24 hours, supernatants were harvested, and cytokines were measured with ELLA. Data are shown from three healthy donors (HDs). FIG. 9B shows a single-cell measure of polyfunctionality (top) and Polyfunctional Strength Index (PSI) (bottom) of CAR T cells stimulated for 4 hours with CD19+ target cells. FIG. 9C shows CAR T cells co-cultured with irradiated 3T3-hCD19 at indicated E:T ratios. The xCELLigence real-time cell analysis (RTCA) system monitored real-time cytotoxicity. FIGS. 9D and 9E show CAR T cells stimulated with irradiated 3T3-hCD19 cells at a 10:1 E:T ratio for 24 hours. Cells were lysed and either total lysate (FIG. 9D) or CAR bound and unbound fractions (FIG. 9E) were western blotted. Data are representative of two HDs (FIGS. 9B-9E). Data are shown as mean±SD. One-way analysis of variance (ANOVA) was performed with Dunnet's multiple comparison test against h19BB06z for FIG. 9A. *p<0.05, **p<0.01, ***p<0.001, ****p<0.0001.

FIGS. 10A to 10F show h19BB06z chimeric antigen receptor (CAR) T cells have greater proliferation, cytotoxicity, and IL-2 production after repeated antigen stimulations with improved in vivo antitumor activity. FIG. 10A shows 5×10⁵ CART cells stimulated with 1×10⁵ irradiated 3T3-hCD19 cells. After 1 week, half of the cells were enumerated by flow cytometry and the other half was re-stimulated with 1×10⁵ fresh irradiated 3T3-hCD19 cells. This was repeated for a total of 4 weeks. FIG. 10B and 10C show after 4 weeks of re-stimulation the same number of CAR T cells for each group was co-cultured with 3T3-hCD19 cells at a 5:1 E:T ratio and either cytotoxicity (FIG. 10B) or cytokine secretion (FIG. 10C) were measured. n=6. Data are representative of 3 (FIG. 10A) or 2 (FIGS. 10B-10C) healthy donors. Data are shown as mean±SD. FIG. 10D shows NOD scid gamma mice were intravenously injected with 5×10⁵ Raji-GFP/luc cells at week −1 and 2×10⁶ CAR T cells were intravenously injected at week 0. Mice were then measured for bioluminescence imaging (BLI) weekly. Average BLI for each week is shown. FIG. 10E shows percentage of Live CAR+cells at week 5. FIG. 10F shows percentage of live CD19 +cells at week 5. n=6. Data are representative of two independent experiments. Two-way analysis of variance (ANOVA) was performed with Dunnet's multiple comparison test against h19BB06z for FIG. 10A. One-way ANOVA was performed with Dunnet's multiple comparison test against h19BB06z for FIG. 10C. **p<0.01, ***p<0.001, ****p<0.0001.

FIGS. 11A to 11C show CD19/CD20 bi-specific constructs are highly expressed and confer chimeric antigen receptor (CAR) T cells cytotoxicity and cytokine production. FIG. 11Ai is a schematic of bi-specific single CAR constructs targeting CD19 and CD20 in a tandem (Tan) configuration. FIG. 11ii is a histogram showing CAR expression. FIG. 11iii shows quantitative analysis of CAR expression (Protein L staining) at day 7 post-transduction compared with 19-28z cells. FIGS. 11Bi to 11Biii show real-time cytotoxicity assay (xCelligence) performed with the different bi-specific CAR T cells and single 19-28z against Raji-CD19^{High}, Raji-CD19^{Low} and Raji-CD19^{KO} target cells at 1:1 E:T ratio. FIGS. 11Ci to 11Ciii show concentration of interferon gamma (IFN-γ), interleukin (IL)-2, IL-6 and tumor necrosis factor alpha (TNF-α) measured by ELLA in supernatants from co-cultures of bi-specific CAR T cells and single 19-28z with Raji-CD19^{High} (FIG. 110i), Raji-CD19^{Low} (FIG. 11Cii), and Raji-CD19^{KO} (FIG. 11Ciii) after 24 hours compared with untransduced T (UT) cells. All conditions were normalized to the lowest CAR expression using UT cells, reaching same number of CAR T cells and total T cells per group. Representative results of three independent experiments are shown. Data are shown as mean±SD. NS, not significant; *p<0.05, **p<0.01, ***p<0.001, ****p<0.0001. One-way analysis of variance (ANOVA) was performed with Dunnet's multiple comparison test against UT.

FIGS. 12A to 11C show bi-specific chimeric antigen receptor (CAR) T cells show different in vivo antitumor efficacy. Nalm6-bearing NSG mice were treated with 1×10⁶ CAR T cells 7 days after initial tumor cell injection. FIG. 12A contains bioluminescent images of each mouse per condition at specified days after T/CAR T cell injection. FIGS. 12Bi to 12Biv show tumor burden (average radiance) of each mouse treated with bi-specific CAR T cells and controls. Two-way analysis of variance (ANOVA) was performed with Dunnet's multiple comparison test against untransduced T cell (UT). Each line represents an individual mouse. FIG. 12C shows Kaplan-Meier analysis of event-free survival defined as time in days to an average luminescence equal or greater than 1×10⁶ p/s/cm2/sr of mice treated with 1×10⁶ T/CAR T cells compared with UT control (n=8 mice per group). *p<0.05.

FIGS. 13A to 13F show CD20-CD19 Tan bi-specific chimeric antigen receptor (CAR) T cells show enhanced in vivo antitumor efficacy and low expression of exhaustion-associated markers. FIG. 13A shows Raji-FFLuc-bearing NSG mice were treated with 1×10⁶ CAR T cells 5 days after initial tumor cell injection. Average tumor burden (average radiance) of mice treated with bi-specific or mono-specific CAR T cells, untransduced T cell (UT) and tumor control. Data are shown as mean±SEM. Two-way analysis of variance (ANOVA) was performed with Dunnet's multiple comparison test against UT (left). Kaplan-Meier analysis of event-free survival defined as time in days to an average luminescence equal or greater than 1×10⁶ p/s/cm2/sr of mice treated with 1×10⁶ T/CAR T cells compared with UT control (right). *p<0.05. FIG. 13B shows total number of CAR T cells per μL of blood of tumor-bearing mice treated with the different CAR T cells (each point is 1 mouse). Two-way ANOVA was performed with Dunnet's multiple comparison test against 19-28z condition. *p<0.05, **p<0.01. FIG. 13C shows total number of CAR T cells per femur of tumor-bearing mice treated with different CAR T cells (each point is 1 mouse). FIG. 13D shows expression of PD1 and CD39 among CAR+cells harvested from the bone marrow of tumor-bearing mice treated with different CAR T cells (n=7 mice per group). Data are shown as mean±SEM. One-way ANOVA was performed with Dunnet's multiple comparison test against the 19-28z condition. FIG. 13E shows RajiCD19^{KO}-bearing NSG mice were treated with 1×10⁶ CART cells 5 days after initial tumor cell injection. Tumor burden (average radiance) of mice treated with bi-specific or mono-specific CAR T cells, UT and tumor control. Each line represents an individual mouse (n=5 mice per group). FIG. 13F shows Kaplan-Meier analysis of survival of mice treated with 1×10⁶ T/CAR T cells compared with tumor only control (n=5 mice per group). P values were determined by a one-sided log-rank Mantel-Cox test. NS, not significant; *p<0.05, **p<0.01. All conditions were normalized to the lowest CAR expression using UT cells, reaching same number of CAR-T cells and total T cells per group.

FIGS. 14A to 14E show 20-19 Tan BB06z bi-specific chimeric antigen receptor (CAR) T cells show increased expansion, memory phenotype and lower exhaustion-associated markers after repeated antigen stimulation. 5×10⁵ CAR T cells were co-cultured with 5×10⁵ target cells (Raji-CD19^{KO}, Raji-CD19^{Low} and Raji-CD19^{High}). After 1 week half the cells were harvested, enumerated and stained by flow cytometry while the other half were re-stimulated with 5×10⁵ fresh target cells (indicated by arrows). This was repeated for a total of 4 weeks. (A) Number of CAR+cells at each week. FIG. 14B shows number of total CD3+cells each week. Two-way analysis of variance (ANOVA) was performed with Dunnet's multiple comparison test when comparing to control group (untransduced T cell (UT)). FIG. 14C shows frequency of central memory (CM—CCR7+CD45RA⁻), naïve (N—CCR7+CD45RA⁺), effector memory (EM—CCR7⁻CD45RA⁻) and terminally differentiated effector (terminally differentiated effector memory cells re-expressing CD45RA (EMRA)—CCR7⁻CD45RA⁺) cells at each week among CAR⁺cells. Representative dot-plot for CCR7/CD45RA staining at each week (right). FIG. 14D shows frequency of PD1⁺CD39⁺ cells within total CAR T cells at each week (top) and within CD8⁺ CAR T cells (bottom). Representative dot-plot for PD1/CD39 staining at each week. FIG. 14E shows after 4 weeks of re-stimulation with Raji-CD19^{High} target cells, the same number of CAR T cells were co-cultured for 24 hours with fresh Raji-CD19^{High} cells at a 1:1 E:T ratio and cytokine secretion was measured. Two-way ANOVA was performed with Sidak's multiple comparison test when comparing 20-19 Tan mut06z to 20-19 Tan BB06z. Data are shown as mean±SEM. NS, not significant; *p<0.05, **p<0.01, ***p<0.001, ****p<0.0001. Data are representative of three healthy donors.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non- target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

Disclosed herein are immune effector cells, such as T cells or Natural Killer (NK) cells, that are engineered to express CAR polypeptides that selectively bind B cell antigens (BCAs). Therefore, also disclosed are methods for providing an anti-tumor immunity in a subject with myeloid or B cell malignancies that involves adoptive transfer of the disclosed immune effector cells engineered to express the disclosed CAR polypeptides.

### Chimeric Antigen Receptors (CAR)

Disclosed herein is a chimeric antigen receptor (CAR) polypeptide that can be that can be expressed in immune effector cells to enhance antitumor activity against myeloid or B cell malignancies.

The disclosed CAR is generally made up of three domains: an ectodomain, a transmembrane domain, and an endodomain. The disclosed bi-specific ectodomain comprises a CD19-binding region and a CD20-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:
SP-CD19VH-CD20VH-LKR-CD20VL-CD19VL-CD8-41BB-CD28-CD3z;
SP-CD20VH-CD19VH-LKR-CD19VL-CD20VL-CD8-41BB-CD28-CD3z;
SP-CD19VL-CD20VL-LKR-CD20VH-CD19VH-CD8-41BB-CD28-CD3z;
SP-CD20VL-CD19VL-LKR-CD19VH-CD20VH-CD8-41BB-CD28-CD3z;
   SP-CD19VH-CD20VH-LKR-CD20VL-CD19VL-CD8-CD28-CD3z;
   SP-CD20VH-CD19VH-LKR-CD19VL-CD20VL-CD8-CD28-CD3z;
   SP-CD19VL-CD20VL-LKR-CD20VH-CD19VH-CD8-CD28-CD3z;
   SP-CD20VL-CD19VL-LKR-CD19VH-CD20VH-CD8-CD28-CD3z;
   SP-CD19VH-CD19VL-CD20VH-CD20VL-CD8-41BB-CD28-CD3z;
   SP-CD19VL-CD19VH-CD20VL-CD20VH-CD8-41BB-CD28-CD3z;
   SP-CD19VH-CD19VL-CD20VL-CD20VH-CD8-41BB-CD28-CD3z;
   SP-CD19VL-CD19VH-CD20VH-CD20VL-CD8-41BB-CD28-CD3z;
   SP-CD20VH-CD20VL-CD19VH-CD19VL-CD8-41BB-CD28-CD3z;
   SP-CD20VL-CD20VH-CD19VL-CD19VH-CD8-41BB-CD28-CD3z;
   SP-CD20VH-CD20VL-CD19VL-CD19VH-CD8-41BB-CD28-CD3z;
   SP-CD20VL-CD20VH-CD19VH-CD19VL-CD8-41BB-CD28-CD3z;
      SP-CD19VH-CD19VL-CD20VH-CD20VL-CD8-CD28-CD3z;
      SP-CD19VL-CD19VH-CD20VL-CD20VH-CD8-CD28-CD3z;
      SP-CD19VH-CD19VL-CD20VL-CD20VH-CD8-CD28-CD3z;
      SP-CD19VL-CD19VH-CD20VH-CD20VL-CD8-CD28-CD3z;
      SP-CD20VH-CD20VL-CD19VH-CD19VL-CD8-CD28-CD3z;
      SP-CD20VL-CD20VH-CD19VL-CD19VH-CD8-CD28-CD3z;
      SP-CD20VH-CD20VL-CD19VL-CD19VH-CD8-CD28-CD3z; or
      SP-CD20VL-CD20VH-CD19VH-CD19VL-CD8-CD28-CD3z;
wherein “SP” represents an optional signal peptide,
wherein “CD19VH” represents an anti-CD19 V_{H} domain,
wherein “CD19VL” represents an anti-CD19 V_{L} domain,
wherein “CD20VH” represents an anti-CD20 V_{H} domain,
wherein “CD20VL” represents an anti-CD20 V_{L} domain,
wherein “LKR” represents a loop linker domain,
wherein “CD8” represents a CD8 hinge domain,
wherein “41BB” represents a 41BB domain,
wherein “CD28” represents a CD28 co-stimulatory signaling region,
wherein “CD3z” represents a CD3 zeta (CD3ζ) region, and
wherein “—” represents a peptide bond or linker.

In some embodiments, the an anti-CD19 V_{H} domain has the amino acid sequence

(SEQ ID NO: 1)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT
or
(SEQ ID NO: 2)
DIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTS
GGGTKLEIK,

In some embodiments, the anti-CD19 V_{L} domain has the amino acid sequence

(SEQ ID NO: 3)
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSAAA
or
(SEQ ID NO: 4)
EVKLQQSGAELVRPGSSVKISCKASGYAFSSYWMNWVKQRPGQGLEWIG
QIYPGDGDTNYNGKFKGQATLTADKSSSTAYMQLSGLTSEDSAVYFCAR
KTISSVVDFYFDYWGQGTTVTVSS,

In some embodiments, the anti-CD20 V_{H} domain has the amino acid sequence

(SEQ ID NO: 5)
EVQLQQSGAELVKPGASVKMSCKASGYTFTSYNMHWVKQTPGQGLEWIGA
IYPGNGDTSYNQKFKGKATLTADKSSSTAYMQLSSLTSEDSADYYCARSN
YYGSSYWFFDVWGAGTTVTVSS,

In some embodiments, the anti-CD20 V_{L} domain has the amino acid sequence

(SEQ ID NO: 6)
DIVLTQSPAILSASPGEKVTMTCRASSSVNYMDWYQKKPGSSPKPWIYAT
SNLASGVPARFSGSGSGTSYSLTISRVEAEDAATYYCQQWSFNPPTFGGG
TKLEIK,

In some embodiments, the CD3 zeta region has the amino acid sequence

(SEQ ID NO: 7)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some embodiments, the 41BB region has the amino acid sequence

(SEQ ID NO: 8)
RGRKKLLYIFKQPFMRPVQTTQEEDGCSCRFPEEEEGGCEL.

In some embodiments, the CD8 hinge domain comprises the amino acid sequence

(SEQ ID NO: 9)
TTTPAPRPPTPAPTIASQPLSLRPEACRPAAGGAVHTRGLDFACDIYIWA
PLAGTCGVLLLSLVITLYC.

In some embodiments, the loop linker domain has the amino acid sequence

(SEQ ID NO: 10)
GSTSGSGKPGSGEGSTKG.

In some embodiments, the CD28 co-stimulatory signaling region comprises a cytoplasmic domain of CD28 having a null mutation in the tyrosine amino acid of the YMNM (SEQ ID NO:11) subdomain, and wherein the co-stimulatory signaling region comprises a cytoplasmic domain of CD28 having a null mutation in the proline amino acids of the PRRP (SEQ ID NO:12) subdomain. In some embodiments, the CD28 co-stimulatory signaling region comprises a cytoplasmic domain of CD28 having a wildtype PYAP (SEQ ID NO:13) subdomain. Therefore, in some embodiments, the CD28 co-stimulatory signaling region comprises the amino acid sequence RSKRSRLLHS DX₁MNMTX₂RRX₃ GPTRKHYQPY APPRDFAAYR S, wherein X1 is not Y, and wherein X₂ and X³ are not P (SEQ ID NO:14), or an amino acid sequence having at least 95% sequence identity to SEQ ID NO:14. In some cases, the X₁, X₂, and X₃ are conservative substitutions. In some embodiments, the CD28 co-stimulatory signaling region comprises the amino acid sequence

(SEQ ID NO: 15)
RSKRSRLLHSDFMNMTARRAGPTRKHYQPYAPPRDFAAYRS.

Additional CAR constructs are described, for example, in Fresnak A D, et al. Engineered T cells: the promise and challenges of cancer immunotherapy. Nat Rev Cancer. 2016 Aug. 23;16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcyRIIIγ, FcεRIβ (FCERIB), and FcεRIγ (FCERIG). Variant CD3ζ signaling domains lacking one or more ITAM domains may also find use in the herein described CARs. Such variant CD3ζ signaling domains include those described by Bridgeman et al., Clin. Exp. Immunol. 175:258-267 (2013), which is herein incorporated by reference.

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD19, -CD20, or -CD22 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the anti-BCA binding agent is single chain variable fragment (scFv) antibody. The affinity/specificity of an anti-BCA scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the anti-BCA binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target a BCA and at least one additional tumor antigen. Also disclosed are CARs designed to work only in conjunction with another CAR that binds a different antigen, such as a tumor antigen. For example, in these embodiments, the endodomain of the disclosed CAR can contain only an signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gpIOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed CARs that allow expression of the CARs in the disclosed immune effector cells. Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DM PG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells”). These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes, preferably cytotoxic T lymphocytes (CTLs).

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8₊ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4₊ or CD8₊. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4₊ T_{reg} cells have been described—naturally occurring T_{reg} cells and adaptive T_{reg} ie cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8₊ T lymphocytes. In some embodiments, the T cells comprise γδ T cells, which possess a distinct T-cell receptor (TCR) having one γ chain and one δ chain instead of α and β chains.

Natural-killer (NK) cells are CD56₊CD3₋ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8₊ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Epstein-Barr virus (EBV)-induced lymphoproliferative diseases (EBV-LPDs) and other EBV-associated cancers are a significant cause of morbidity and mortality for recipients of allogeneic hematopoietic cell transplantation (HCT) or solid organ transplants (SOT), particularly in those who have received certain T-cell reactive Abs to prevent or treat GVHD. Prophylaxis and treatment by the adoptive transfer of autologous or allogeneic EBV-specific cytotoxic T cells and the subsequent long-term restoration of immunity against EBV-associated lymphoproliferation have provided positive outcomes in the management of these uniformly fatal complications of allogeneic tissue transfer. Therefore, in some embodiments, the disclosed immune effector cells that comprise one or more of the CAR polypeptides of the present invention are allogeneic or autologous EBV-specific cytotoxic T lymphocytes (CTLs). For example, generation of EBV-specific cytotoxic T cells may involve isolating PBMCs from of an EBV-seropositive autologous or allogenic donor and enriching them for T cells by depletion of monocytes and NK cells. EBV-specific cytotoxic T cells may also be produced by contacting donor PBMCs or purified donor T cells with a “stimulator” cell that expresses one or more EBV antigen(s) and presents the EBV antigen(s) to unstimulated T cells, thereby causing stimulation and expansion of EBV-specific CTLs. EBV antigens include, for example, latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. Cytotoxic T cells that comprise T cell receptor(s) which recognize one or more EBV-specific antigens are deemed to have been “sensitized” to those EBV antigen(s) and are therefore termed “EBV-sensitized cytotoxic T cells” herein. Known methods for generating allogeneic or autologous EBV-specific cytotoxic T cell populations that may comprise one or more of the CAR polypeptides of the present invention are described, for example, in Barker et al., Blood 2010 116(23):5045-49; Doubrovina, et al., Blood 2012 119(11):2644-56; Koehne, et al. Blood 2002 99(5):1730-40; and Smith et al. Cancer Res 2012 72(5):1116-25, which are incorporated by reference for these teachings.

### Therapeutic Methods

Immune effector cells expressing the disclosed CARs can elicit an anti-tumor immune response against CD19-, CD20-, and/or CD22-expressing cancer cells. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to CD19, CD20, and/or CD22.

Adoptive transfer of immune effector cells expressing chimeric antigen receptors is a promising anti-cancer therapeutic. Following the collection of a patient's immune effector cells, the cells may be genetically engineered to express the disclosed CD19-, CD20-, and/or CD22-specific CARs, then infused back into the patient. Moreover, immune effector cells obtained from a donor other than the patient (i.e., allogeneic to the patient) may be genetically engineered to express the disclosed CD19-, CD20-, and/or CD22-specific CARs, then the CAR-containing cells infused into the patient. In one specific embodiment, the immune effector cells are allogeneic EBV-specific cytotoxic T cells.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any CD19-, CD20-, and/or CD22-expressing cell in a subject undergoing unregulated growth, invasion, or metastasis. Cancers that express CD19, CD20, or CD22 include prostate cancer, ovarian cancer, adenocarcinoma of the lung, breast cancer, endometrial cancer, gastric cancer, colon cancer, and pancreatic cancer. CD19, CD20, or CD22 has also been found on Jurkat cells. In some aspects, the cancer is a gallbladder cancer, exocrine adenocarcinoma, or apocrine adenocarcinomas.

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Techniques for combining CARs with checkpoint inhibitors in immune effector cells and use thereof for the treatment of various disorders are described, for example, in WO 2017/040945, which is incorporated by reference herein.

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAI L)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy- progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

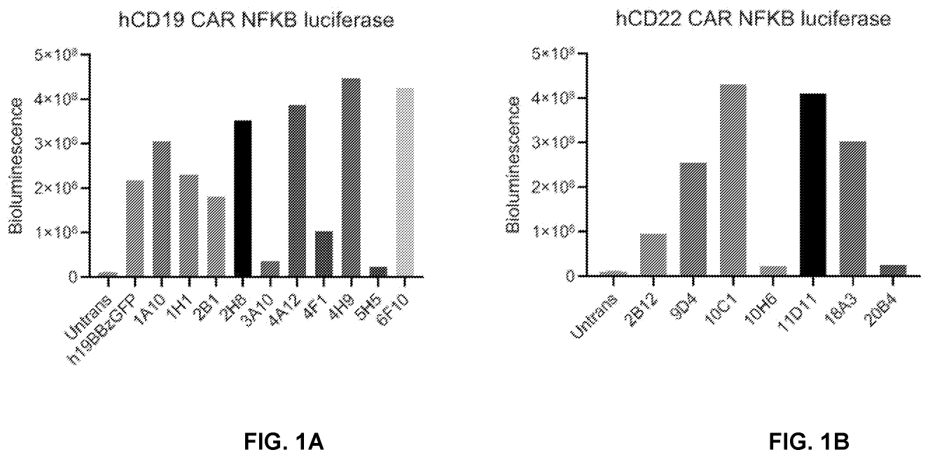

FIG. 1. Human CD19 CARs and human CD22 CARs induce NF-κB in NF-κB293 reporter cells. NF-κB293 reporter cells stably express a transgene, in which NF-κB responsive transcriptional elements are placed upstream of a minimal CMV-GFP-luciferase cassette. So, luciferase activity reflects activity of NF-κB signaling pathway. CAR constructs were packaged into recombinant retrovirus, which was used to transduce NF-κB293 reporter cells. Forty-eight hours later, cell lysates were prepared from transduced reporter cells and untransduced controls. NF-κB activation was evaluated using luciferase assay (FIGS. 1A and 1B).

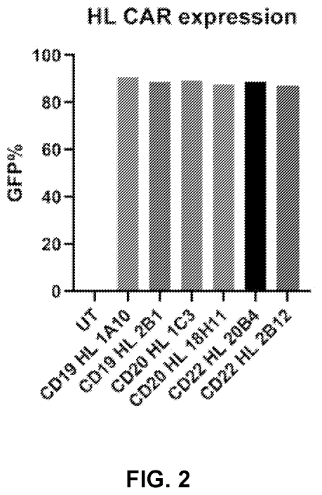

CAR expression in human CAR-T cells. On day 0, human T cells were isolated from healthy donor PBMCs and activated with human CD3/CD28 dynabeads. T cells were spin transduced retrovirally with CARs on day 1 and day 2. On day 3, fresh medium with IL2 were added. On day 4, CAR-T cells were harvested, de-beaded and evaluated by flow cytometry (FIG. 2). All CD19, CD20 or CD22 CARs tested were tagged with GFP. GFP % reflects CAR expression. Hybridoma cell IDs were indicated.

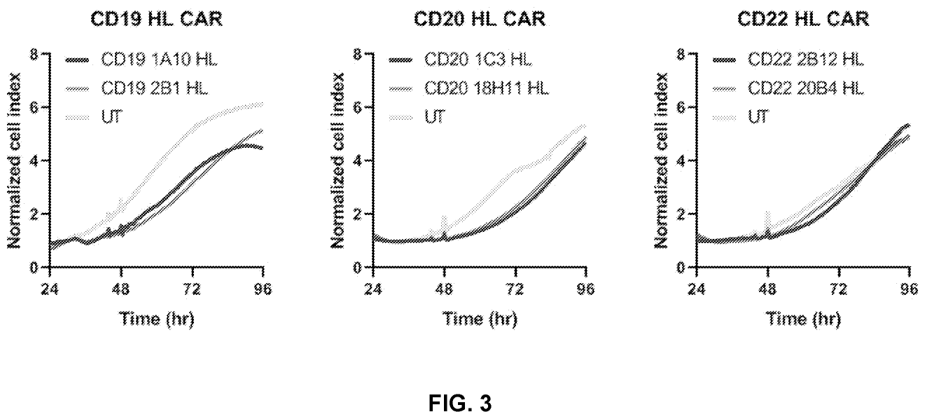

Human CAR T cells were produced and co-cultured with target cells at E:T ratio of 10:1. Target cell killing was monitored on an xCELLigence RTCA system (FIG. 3). Normalized cell index reflects cell growth.

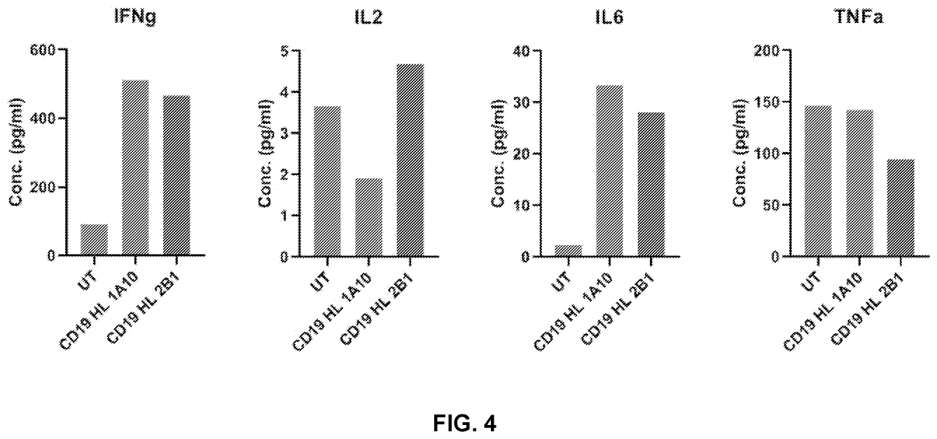

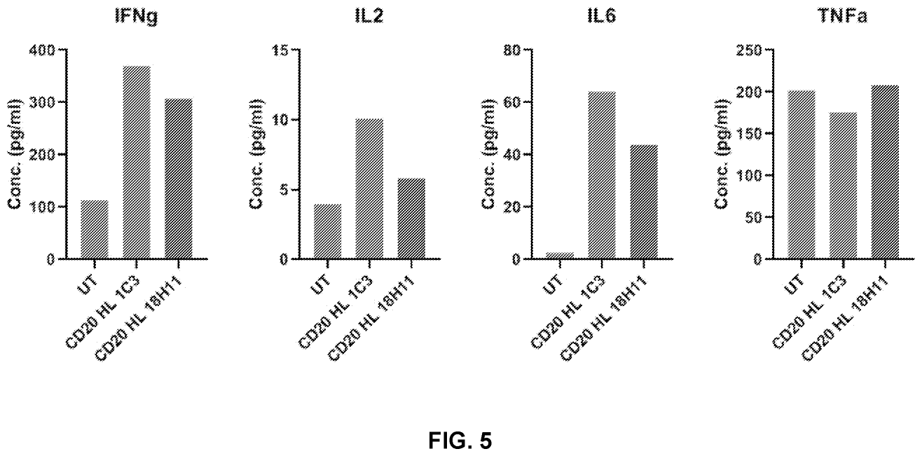

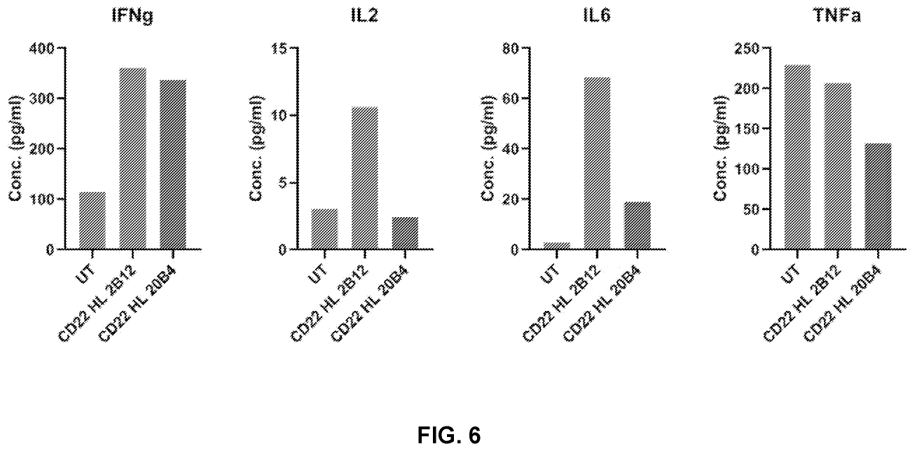

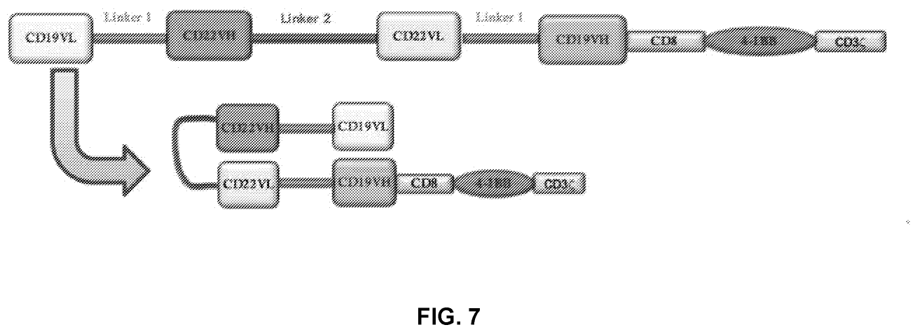

Human CAR T cells were produced and co-cultured with target cells at E:T ratio of 10:1. Twenty-four hours later, supernatant was collected and subjected to ELISA analysis for CD19 (FIG. 4), CD20 (FIG. 5), and CD22 (FIG. 6) using an Ella machine

### Example 2

### Materials and Methods

### Cells

NIH/3T3 cells retrovirally transduced with human CD19 were used as target cells. NIH/3T3 cells were purchased from ATCC. CHO cells retrovirally transduced with human CD20 were used as target cells. CHO cells were purchased from ATCC. K562 cells retrovirally transduced with both human CD19 and CD20 were used as target cells. Nalm6 and RajiWT cells were transduced to express green fluorescent protein (GFP)-firefly luciferase (FFLuc). Raji CD19 antigen panel cell lines which include three genetically modified cell lines involving the CD19 gene (Raji-CD19^{KO}, Raji-CD19^{Low}, and Raji-CD19^{High}) were purchased from Sigma-Aldrich and used as target cells. These three cells lines were l also retrovirally transduced to express GFP-nanoluciferase (NanoLuc) for their in vivo detection. Human peripheral blood mononuclear cells (PBMCs) were purchased from AllCells. T cells were enriched from PBMCs using the EasySep human T cell isolation kit according to manufacturer's instructions (STEMCELL). Human T cell complete medium consists of RPMI1640 medium, 10% fetal bovine serum, 2 mM L-glutamine, 100 U/mL penicillin, and 100 μg/mL streptomycin. All media and supplements were purchased from ThermoFisher Scientific. Cell lines were authenticates as previously described (Boucher J C, et al. Cancer Immunol Res 2021 9:62-74) and used at low passages (3 to 6). Cells were tested for Mycoplasma using the Universal Mycoplasma Detection Kit (ATCC) and were negative.

### Genetic Constructs and CAR T Cell Production

The SFG retroviral backbone was modified to include the FMC63 single-chain variable fragment (ScFv) with CD8α transmembrane and hinge domain followed by one or more co-stimulatory domains and CD3ζ for all constructs. The h1928z, h19BBz, and h1906z CAR constructs have been described (Boucher J C, et al. Cancer Immunol Res 2021 9:62-74). Mut06 replaces YMNM (SEQ ID NO:11) and PRRP (SEQ ID NO:12) CD28 subdomains with FMNM (SEQ ID NO:16) and ARRA (SEQ ID NO:17). The h1906BBz construct has the mut06 co-stimulatory domain proximal to the cell membrane followed by 4-1BB and CD3ζ. The h19BB06z construct has the 4-1BB co-stimulatory domain proximal to the cell membrane followed by mut06 and CD3ζ. Bi-specific constructs were included into the SFG retroviral backbone comprising a CD8α leader peptide followed by a scFv specific for human CD19 (FMC63) linked to a scFv specific for human CD20 (Leu16) engineered in a tandem configuration followed by a CD8α transmembrane domain and either mut06 or 4-1BB plus mut06 and CD3ζ. All plasmids were synthesized externally (Genewiz) and verified by restriction enzyme digest.

All SFG constructs were calcium phosphate transfected into H29 cells. Retroviral supernatants of transfected H29 were harvested and used to transduce RD114 cells. Retroviral supernatant of producer cells was harvested, 0.45 μm filtered, and used to transduce T cells as described (Davila M L, PLoS One 2013 8:e61338; Li G, et al. Methods Mol Biol 2017 1514:111-8). Viability was measured by trypan blue staining and enumerated on an automated cell counter (Bio-Rad). Transduction efficiency was estimated by flow cytometry as a percentage of protein L+live cells. For downstream experiments, CAR T cell doses were normalized based on CAR gene transfer but not sorted to exclude CAR-negative T cells. As a result, the total T cell dose was varied. For all experiments using bi-specific constructs cells were normalized to the lowest CAR expression by accordingly adding varying quantities of untransduced T (UT) cells, reaching the same number of CAR+cells and total T cells per group.

### Mice and Systemic Tumor Models

Female and/or male mice at 8-12 weeks of age were used. NOD scid gamma (NSG) mice (NOD.Cg-Prkdc^{scid} II2rg^{tm1WjI/SzJ}) were purchased from the Jackson Laboratory and bred in our facility. Mice were intravenously injected with 5×10⁵ Raji-GFP/FFluc or Nalm6-GFP/FFluc cells at week −1 and 2×10⁶ CAR T cells were intravenously injected at week 0. For bi-specific CAR T cells mice received 1×10⁶ CART cells. Tumors were measured using an IVIS Lumina III In Vivo Imaging System (Perkin-Elmer) for bioluminescence imaging (BLI) weekly. Mice were monitored for illness daily and sacrificed when there was evidence of leukemia progression, such as decreased activity, hunched posture, or ruffled coat. At certain time points, blood was collected from the submandibular vein into tubes containing K3 EDTA (Sarstedt). At mouse sacrifice blood, spleen, and/or femurs were collected. For blood samples red blood cells were lysed using ammonium-chloride-potassium buffer (ThermoFisher) and stained for flow cytometry as described below. Bone marrow was isolated from femurs by cutting both ends of the femur and flushing it with a syringe containing phosphate buffered saline (PBS) (Gibco). These cells were passed through a 70 pm cell strainer. Red blood cells were lysed as described above and stained for flow cytometry (as described below).

### Flow Cytometry

The following antibodies with clones listed were obtained from BD Biosciences: anti-hCD3 (HIT3a). The following antibodies were from BioLegend: anti-hCD3 (HIT3a), anti-hCD19 (HIB19), anti-hPD1 (NAT105), anti-hCD45RA (HI100), anti-hCCR7 (G043H7), anti-hCD4 (OKT4), anti-hTIM3 (F38-2E2), anti-CD39 (A1), anti-hCD20 (2H7), anti-hCD45 (HI30), anti-hCD8 (RPA-T8), Streptavidin conjugated with PE/Cyanine7. Streptavidin conjugated with Alexa Fluor 488, Biotinylated Recombinant Protein L, Fixable Viability Dye eFluor 450 were purchased from ThermoFisher Scientific.

Cells were first washed twice with PBS and stained with a fixable viability dye (eBioscience) at room temperature for 30 min. Surface staining was performed at 4° C. with Fc block and antibody mix in magnetic-activated cell sorting buffer with 0.5% bovine serum albumin (BSA) (Miltenyi Biotec). For some experiments, Countbright beads (ThermoFisher Scientific) were used for cell quantification following manufacturer's instructions. All samples were analyzed with a 5-laser LSRII (BD Biosciences), and data were analyzed using FlowJo software (Tree Star).

### Cytokine Immunoassay

1×106 or 5×104 CART cells were washed and co-cultured with different ratios of target cells for 24 hours. Supernatants were collected and analyzed using an ELLA® Assay kit (Multianalyte: interferon gamma (IFN-γ), interleukin 2 (IL-2), IL-6 and tumor necrosis factor alpha (TNF-α)) according to the manufacturer's instructions.

### Cytotoxicity Assay

Cytotoxicity assays were run on an xCelligence real-time cell analysis (RTCA) instrument (ACEA Biosciences) according to the manufacturer's instructions. Briefly, 1×104 adherent target cells (3T3-hCD19 or CHO-hCD20) were plated per well on an E-Plate 96. For non-adherent cells (Raji and K562) tethering antibodies were used to coat xCelligence E-Plate 96 wells according to the manufacturer's instructions. Briefly, xCelligence E-Plate 96 were coated with anti-CD40 (Raji) or anti-CD71 (K562) antibodies for 3 hours at room temperature then washed and 3×10⁴ target cells were added per well. The next day CAR T cells were re-suspended in fresh complete medium without IL-2 and added onto target cells at various effector to target (E:T) ratios, and growth was monitored.

### Isolight

After experimental design and prior to plating cells into chips, CAR T cells were stimulated for 4 hours or overnight with target cells at a 5:1 E:T ratio. For certain experiments total CAR T cells were plated and analyzed in an IsoLight instrument and for others magnetically isolated CD4+ and CD8+ cells were plated independently. Polyfunctionality: co-secretion of 2+ cytokines per cell; Polyfunctional Strength Index (PSI): percentage of polyfunctional cells in the sample multiplied by the intensities of secreted cytokines.

### Western Blot

A total of 1×10⁷ CAR T cells per CAR were stimulated with 3T3-hCD19 target cells for 24 hours or left unstimulated. Cells were lysed with radioimmunoprecipitation assay buffer and total protein was quantified by ND1000 (Nanodrop); 20 μg of lysate was loaded onto the gel. Proteins were detected using anti-phospho-lymphocyte-specific protein tyrosine kinase (pLCK) (1:1000 5% BSA; phospho Tyr505; Cell SignaliNg), anti-lymphocyte-specific protein tyrosine kinase (LCK) (1:2000 5% BSA; BD), and anti-glyceraldehyde 3-phosphate dehydrogenase (1:5000 5% BSA; Cell SignaLing). Goat antirabbit IgG (H+L) and goat antimouse IgG (H+L) (Cell Signaling) were used as secondary antibodies. The secondary antibodies were diluted in 5% BSA at a 1:10 000 dilution. Primary antibodies were incubated at 4° C. overnight, and secondary antibodies were incubated at room temperature for 2 hours. ChemiDocMP Imaging System (Bio-Rad) was used to detect enhanced chemiluminescence (Pierce ECL Western Blotting Substrate) western blotting signals.

### Statistics

All statistical analyses were conducted using Prism V.8 software (GraphPad). No statistical methods were used to predetermine sample size. Survival was compared using a log-rank test. Values of p≤0.05 were considered significant. The statistical tests used for each experiment are described in each figure legend.

### Results

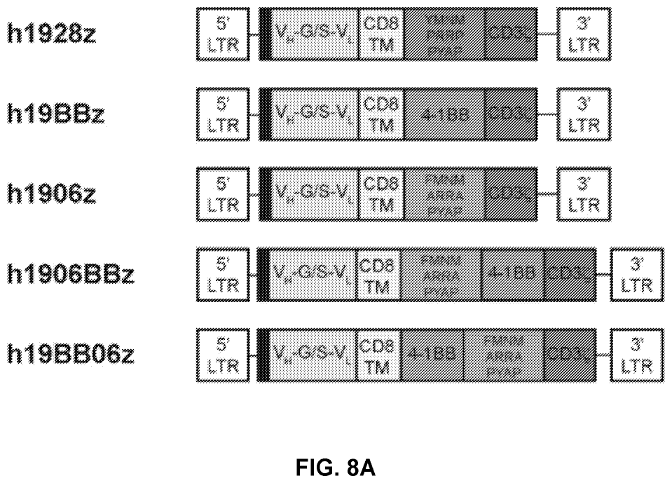

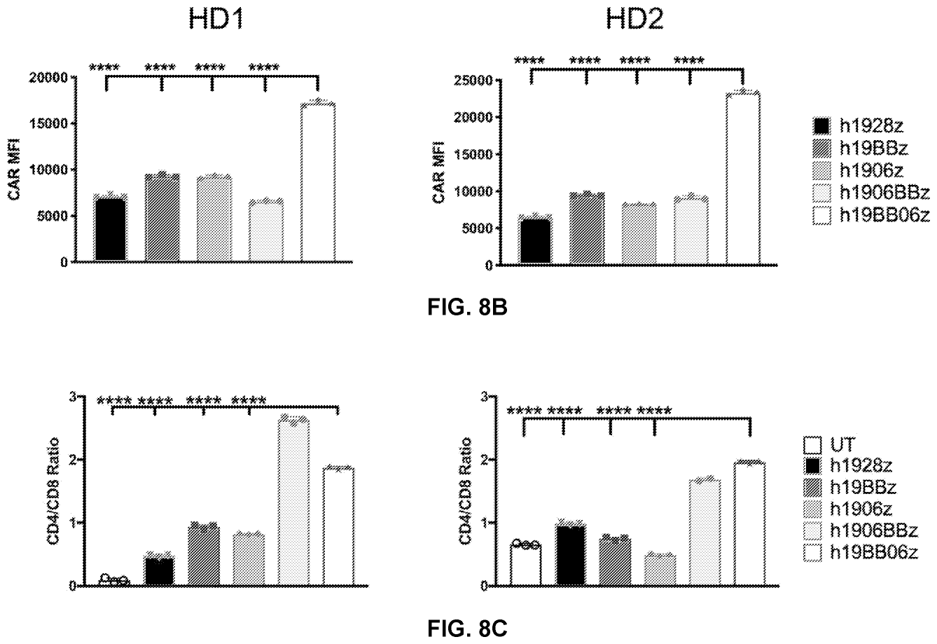

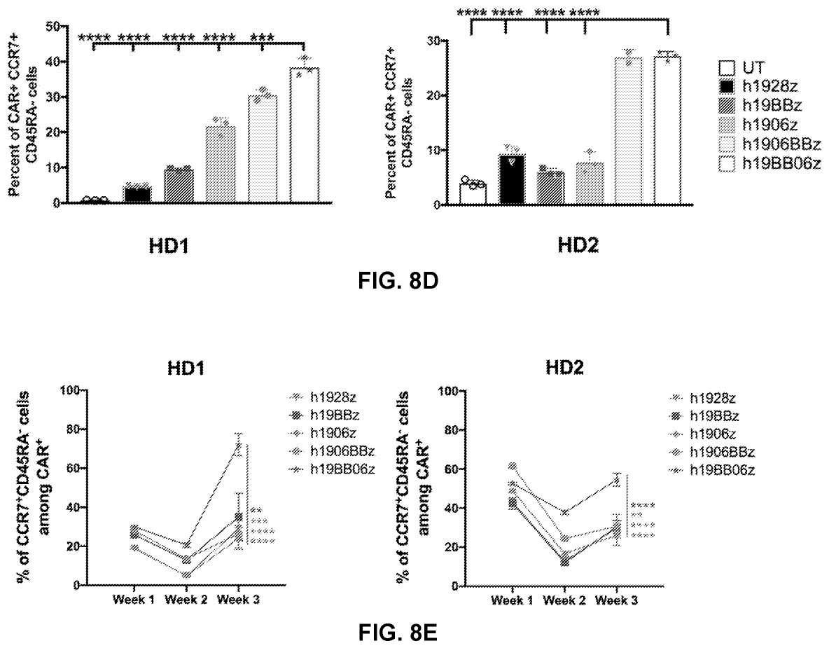

Co-Stimulation With 4-1BB and mut06 Results in a Favorable Memory Phenotype To evaluate how the combination of 4-1BB and mut06 co-stimulation affects CAR function, five human CD19-targeted CARs were designed (FIG. 8A). All CAR constructs include the FMC63 scFv with a CD8α transmembrane and hinge domain followed by the co-stimulatory domain/s and CD3ζ. h1928z, h19BBz, and h1906z (mut06) have been described previously (Boucher J C, et al. Cancer Immunol Res 2021 9:62-74). h1906z replaces the CD28 subdomains of YMNM (SEQ ID NO:11) and PRRP (SEQ ID NO:12) with FMNM (SEQ ID NO:16) and ARRA (SEQ ID NO:17), leaving only a functional PYAP (SEQ ID NO:13) signaling motif. h1906BBz includes the mut06 domain proximal to the cell membrane followed by 4-1BB and CD3ζ while h19BB06z has 4-1BB proximal to the cell membrane followed by mut06 and CD3ζ. After retroviral transduction and prior to antigen exposure, it was found that all CAR T cells had similar proliferation and viability and that CARs with multiple co-stimulatory domains showed higher transduction efficiency, with h19BB06z showing the highest CAR surface expression prior to antigen stimulation. CARs combining 4-1BB and mut06 showed higher CD4/CD8 ratios before CAR engagement.

To determine the effect these CARs have on T cell phenotype, CAR T cells were stimulated at a 1:10 E:T ratio for 24 hours with hCD19-expressing target cells (3T3-hCD19). After stimulation, cells were analyzed by flow cytometry and found the highest surface CAR expression on h19BB06z CAR T cells compared with other constructs, same as before CAR engagement (FIG. 8B). The skew towards CD4⁺ CAR T cells was also maintained after CAR stimulation in 4-1BB/mut06-containing CARs (FIG. 8C).

Previous work has shown that CAR T cells with a less differentiated phenotype such as CM or naïve display better expansion, persistence, and antitumor activity in vivo (Gattinoni L, et al. J Clin Invest 2005 115:1616-26; Crompton J G, et al. Immunol Rev 2014 257:264-76; Gattinoni L, et al. Blood 2013 121:567-8; Lugli E, et al. J Clin Invest 2013 123:594-9). Staining with memory markers after 24 hours of antigen stimulation revealed that CARs combining 4-1BB and mut06 had an increased percentage of CM (CCR7⁺CD45RA⁻) CAR T cells compared with other CARs (FIG. 8D). To further validate that our observations after 24 hours of antigen stimulation reflected cell differentiation at longer time points, CAR T cells were stimulated with CD19-expressing cells weekly for a total of 3 weeks and evaluated their phenotype by flow cytometry each week. It was determined that h19BB06z CAR T cells were able to maintain a significantly higher frequency of CM cells while the rest skewed towards a more differentiated phenotype (FIG. 8E). Together these data show that h19BB06z CAR T cells have a more favorable memory-like phenotype compared with other constructs.

4-188 and mut06 Co-Stimulatory Domains Influence Different Aspects of h190688z and h198806z CAR T Cell Function

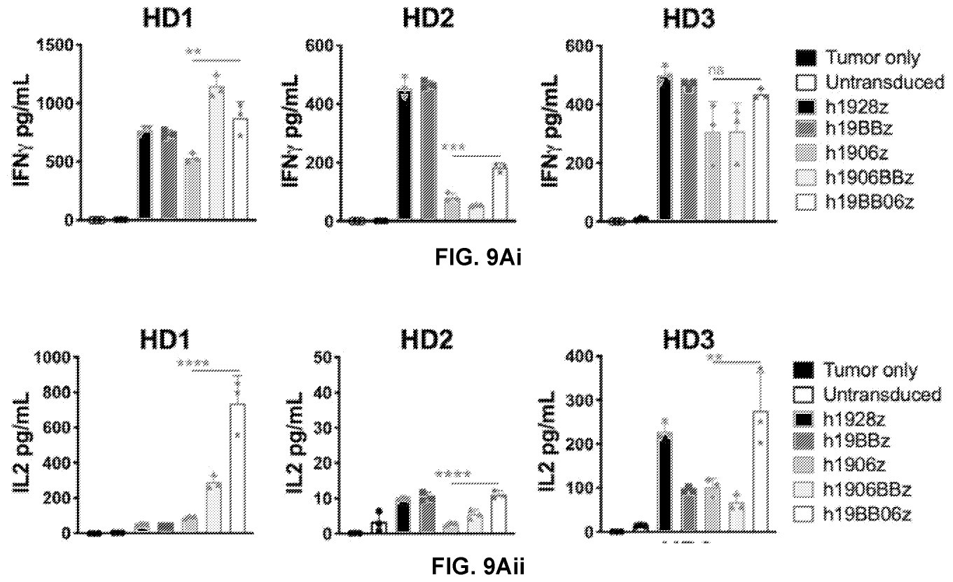

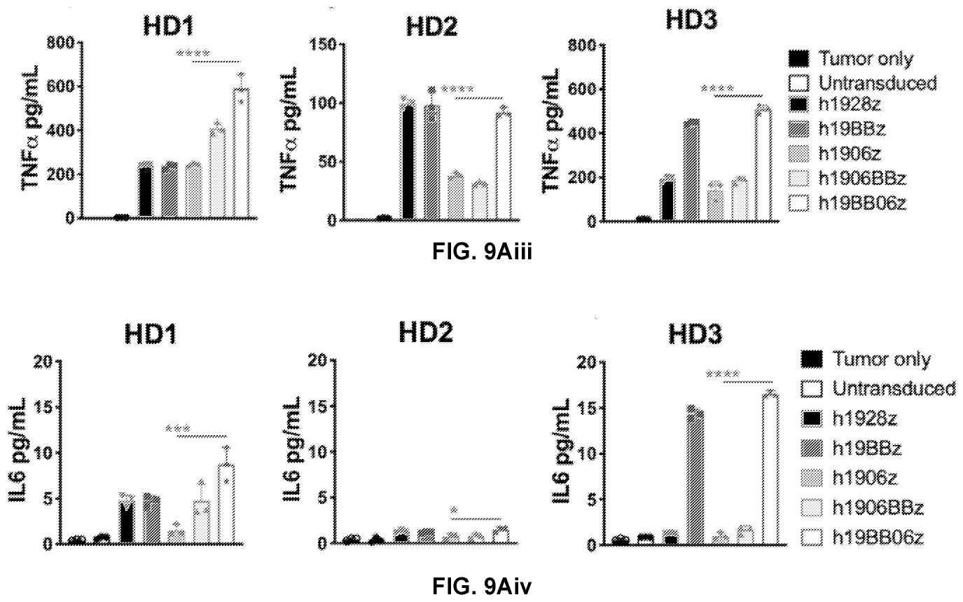

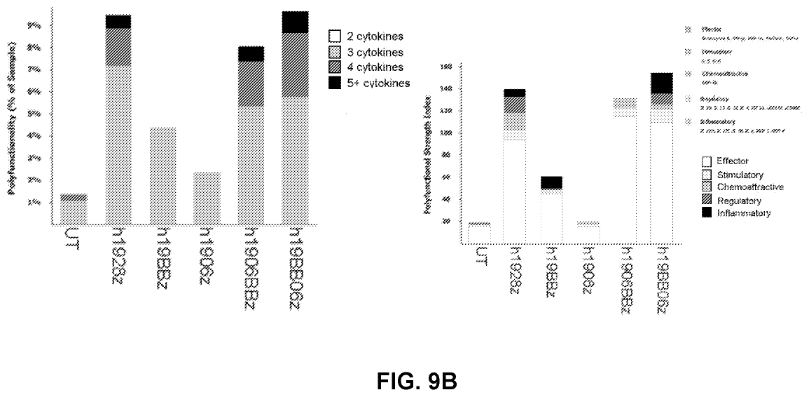

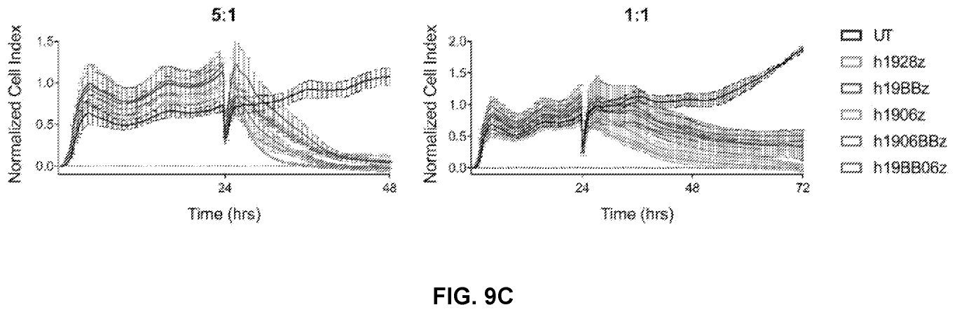

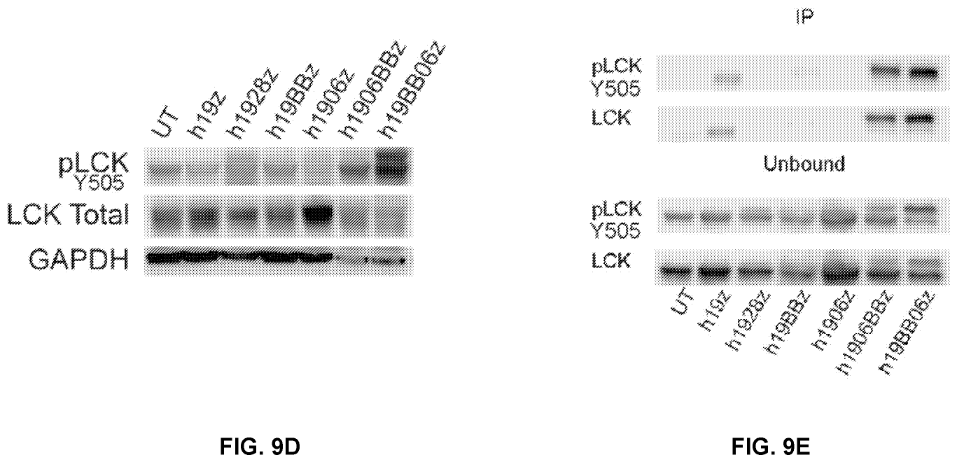

To examine the effector function of the different CARs, cytokine secretion was quantified after 24 hours in vitro stimulation with target cells at a 10:1 E:T ratio. Using three healthy donors it was found that h1906z had the lowest levels of IFN-γ, IL-2 and TNFα compared with all other constructs, which is consistent with the dampening of CD28 signaling by loss of function mutations (Boucher J C, et al. Cancer Immunol Res 2021 9:62-74). When 4-1BB was combined with mut06 we observed an increase in the secretion of IFN-γ, IL-2 and TNF-α, specifically in the configuration where 4-1BB is followed by mut06 (h19BB06z) (FIG. 9A). IL-6 secretion, associated with cytokine release syndrome in patients (Faramand R, et al. Clin Cancer Res 2020 26:4823-31), was minimal in all constructs although highest in h19BB06z. In line with these observations, CAR T cell polyfunctionality was studied, defined as the ability of each individual cell to secrete two or more cytokines. Several recent reports have demonstrated CAR T cell polyfunctionality is a substantial predictor of in vivo fitness (Fousek K, et al. Leukemia 2021 35:75-89; Schmidts A, et al. Blood Adv 2019 3:3248-60; Rossi J, et al. Blood 2018 132:804-14). To examine this, a multiplexed antibody-coated chip was used that analyzes hundreds of CAR T cells at the single-cell level for frequency and intensity of 28 secreted cytokines. CAR T cells were stimulated for 4 hours with target cells before loading onto chips and analyzed. It was found that h1928z, h1906BBz, and h19BB06z had an increased percentage of polyfunctional CAR T cells (FIG. 9B). The PSI is the percentage of polyfunctional cells multiplied by the intensity of the secreted cytokines. Using this measure, h1928z, h1906BBz, and h19BB06z showed the highest PSI (FIG. 9B). This demonstrates that combining 4-1BB and mut06 enhances CAR T cell polyfunctionality, which was similar to h1928z.

In vitro killing ability was then examined by using a real-time cytotoxicity assay with two different E:T ratios (5:1 and 1:1). All CAR constructs had rapid and efficient killing at both ratios (FIG. 9C). This suggests that in vitro cytotoxicity is not greatly affected by choice or combination of co-stimulatory domain.

Lymphocyte-specific protein tyrosine kinase (LCK) is a critical molecule for CAR T cell function. Recently it has been shown that recruitment of LCK to 4-1BB CARs can also enhance CAR T cell cytotoxicity (Sun C, et al. Cancer Cell 2020 37:216-25). The effect of continuous antigen exposure on LCK phosphorylation after 24 hours stimulation was examined. Both third-generation CARs had increased pLCK compared with second-generation CARs (FIG. 9D). Overall, h19BB06z had the highest LCK phosphorylation, suggesting that the co-stimulatory domains' positioning may affect pLCK. It was also found that large amounts of pLCK and total LCK were associated with the h19BB06z CAR molecule (FIG. 9E). These data suggest that the enhanced in vitro function of h1906BBz and h19BB06z results from increased pLCK and that the orientation of the co-stimulatory domains can affect CAR-associated LCK signaling.

### Co-Stimulatory Domain Positioning Affects In Vitro Expansion and In Vivo Tumor-Killing

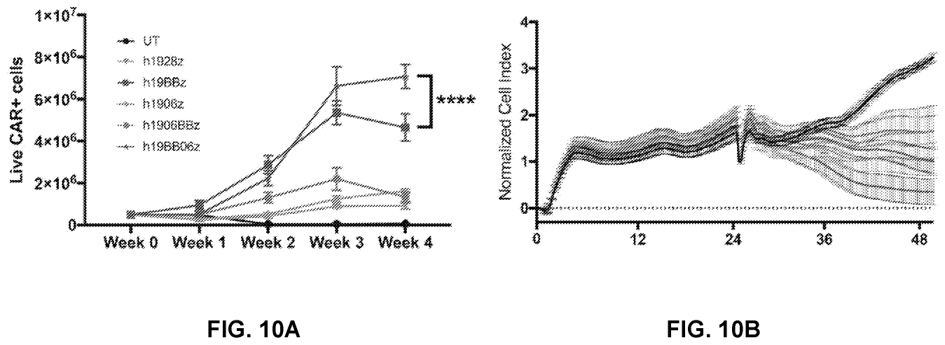

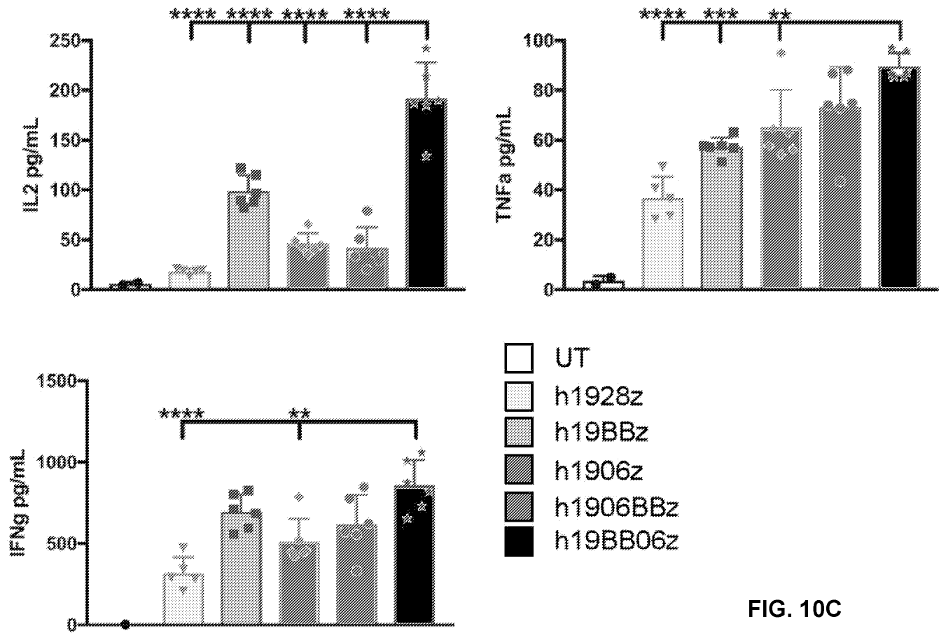

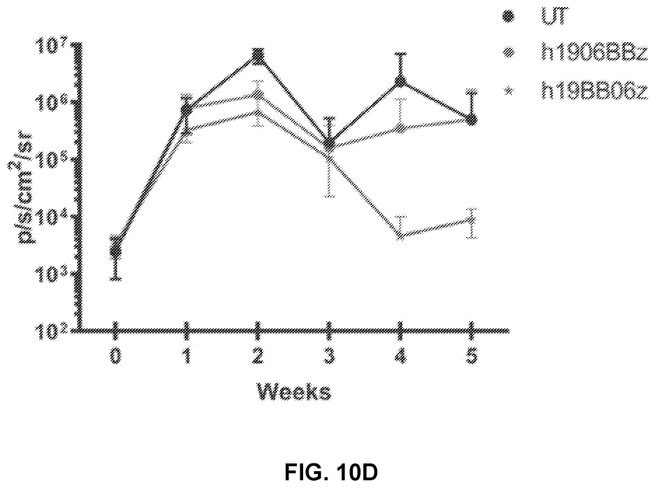

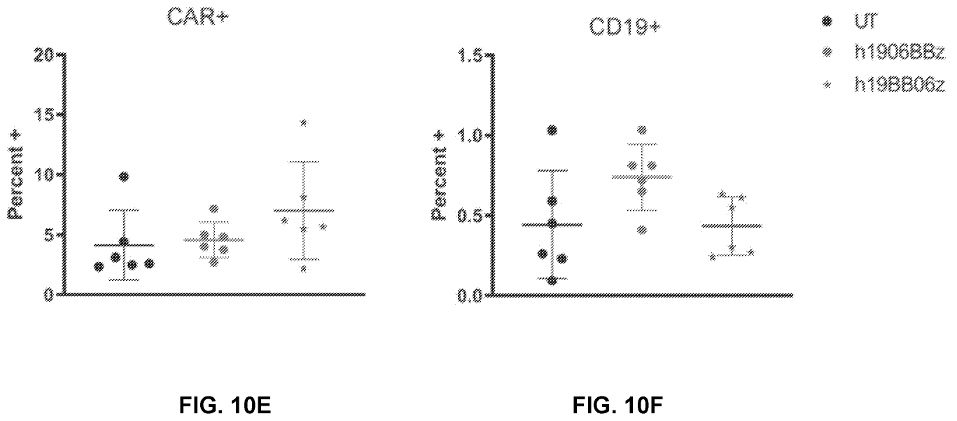

One measure of CAR T cell fitness is its ability to withstand multiple rounds of antigen-stimulation (Cherkassky L, et al. J Clin Invest 2016 126:3130-44). To examine this, CAR T cells were stimulated with target cells at a 5:1 E:T ratio. Re-stimulation of CAR T cells with target cells occurred every 7 days for a total of 4 weeks. A significantly greater expansion of h19BB06z CAR T cells compared with all other groups was observed (FIG. 10A). To study the function of these cells that have endured multiple antigen challenges, their cytotoxicity and cytokine secretion was examined. To control for the enhanced proliferation of h19BB06z, the same number of CAR+ T cells for each group was used in these experiments. h19BB06z had the greatest killing ability compared with all other groups (FIG. 10B). h19BB06z also showed significantly higher secretion of IL-2 compared with all other groups (FIG. 10C). Similarly, h19BB06z displayed significantly greater secretion of effector cytokines TNF-α and IFN-γ compared with h1928z and h1906z (FIG. 10C). h1906BBz CAR T cells displayed lower proliferation, cytotoxicity, and IL-2 production than h19BB06z, again suggesting that co-stimulatory domain positioning can affect CAR function. These data show that after repeated antigen stimulations h19BB06z CAR T cells exhibit the greatest proliferative capacity, cytotoxicity and cytokine secretion, in contrast to single antigen stimulation when the h1928z CAR had the greatest cytokine production and cytotoxicity.

To determine the ability of h1906BBz and h19BB06z CAR T cells to control tumor growth in vivo, Raji B lymphocyte tumor cells were injected into NSG mice. It was found that h19BB06z CAR T cells were better able to control tumor growth compared with UT and h1906BBz (FIG. 10D). In the blood of mice receiving h19BB06z there was a slight increase of CAR+ cells (FIG. 10E) and a corresponding decrease in CD19+ cells (FIG. 10D) compared with h1906BBz. Together, these data show that h19BB06z CAR T cells have enhanced in vitro proliferation, long-term cytotoxicity, and in vivo tumor-killing compared with h1906BBz. Therefore, the BB06 orientation was selected for further evaluation.

### Bi-specific CAR T Cells Targeting CD19 and CD20 Show High Surface CAR Expression With Efficient In Vitro Cytotoxicity and Cytokine Production

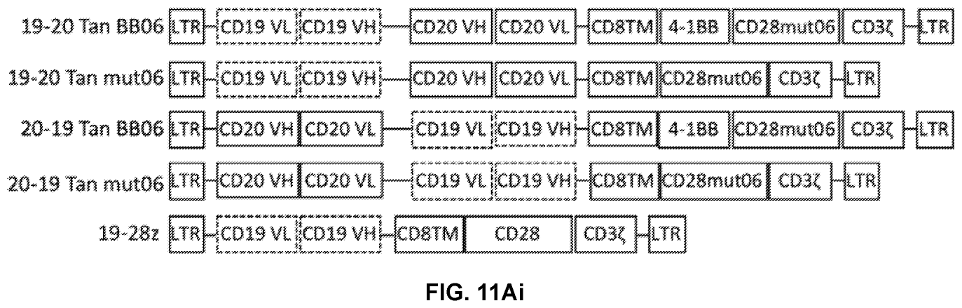

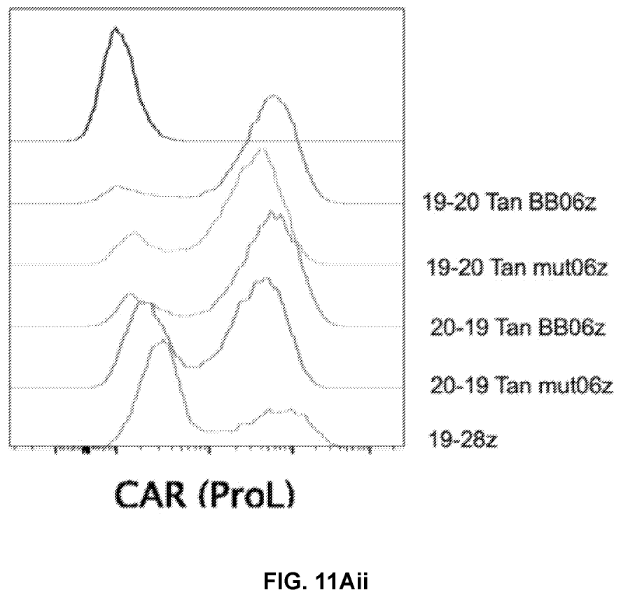

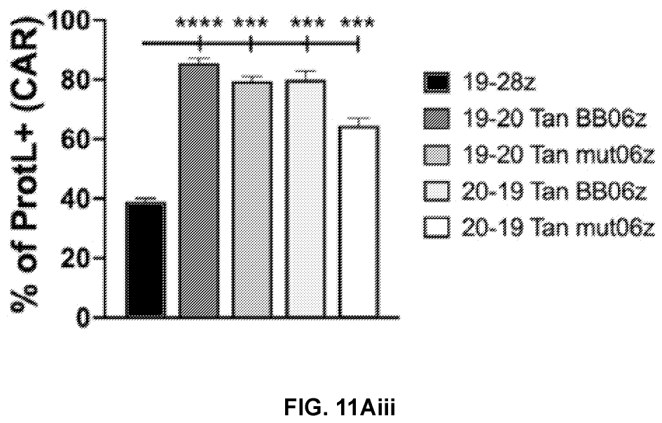

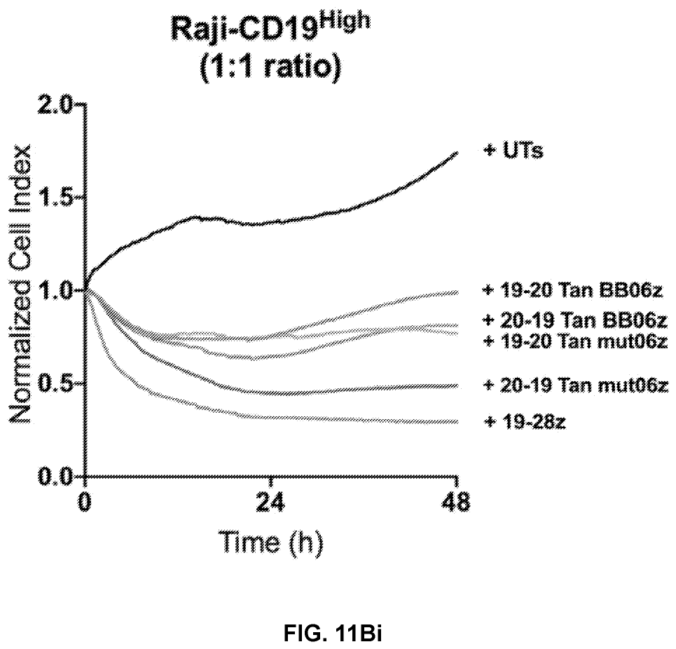

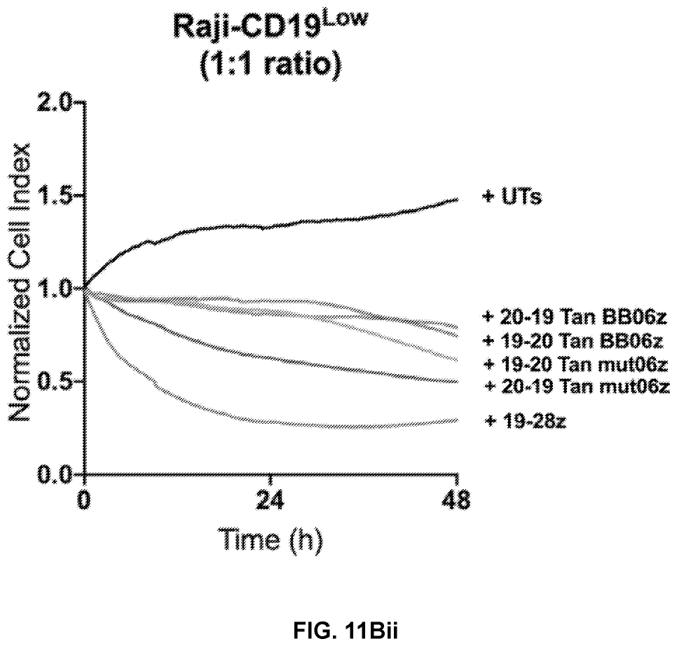

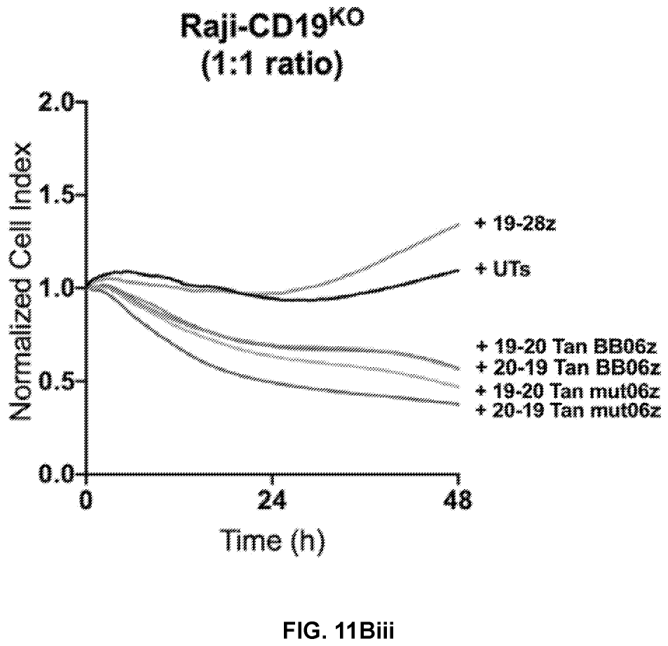

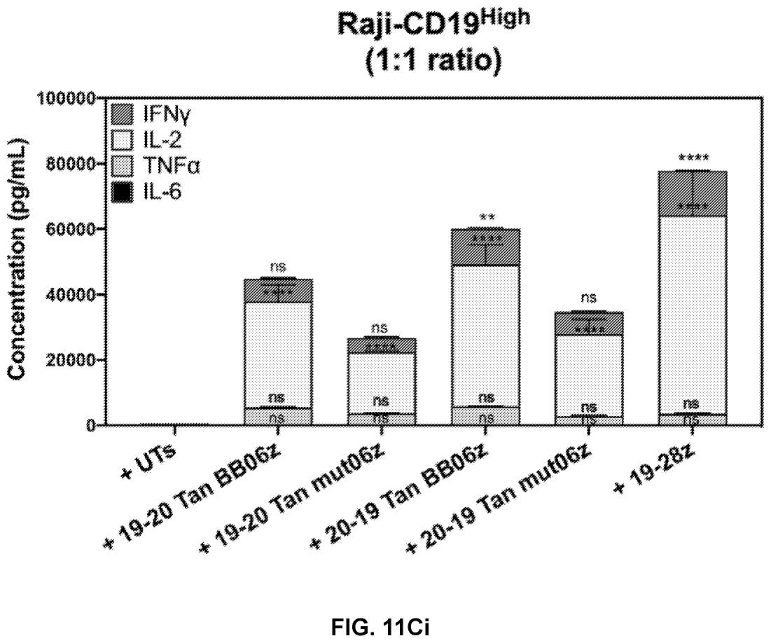

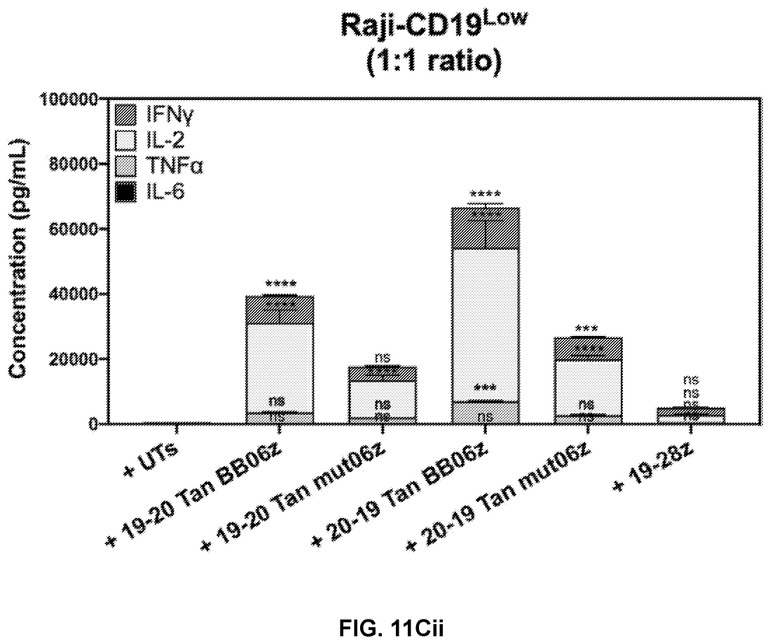

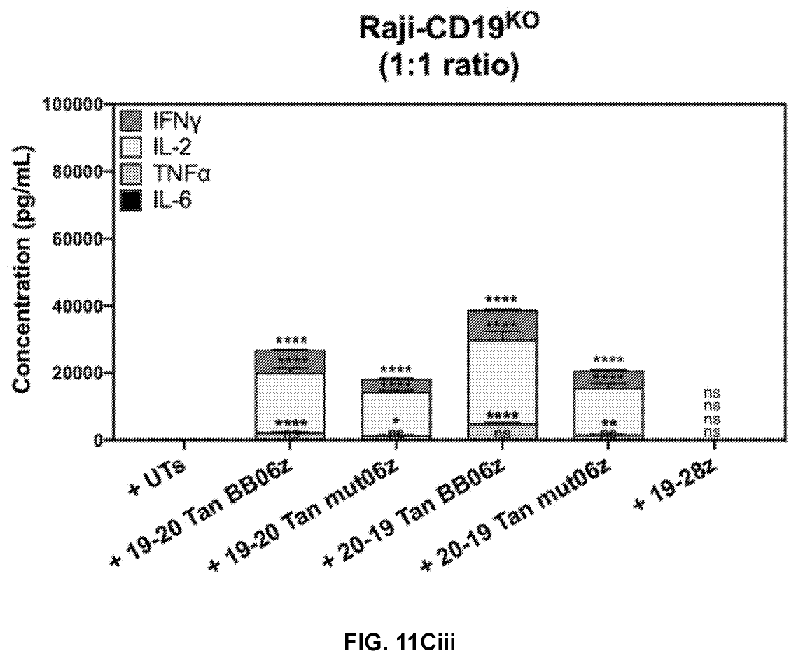

To further validate observations and capitalize on dual-antigen recognition constructs were developed encoding for single bi-specific CAR molecules able to recognize both human CD19 and CD20. This multiantigen recognition approach is shown to mitigate disease relapse due to antigen escape (Majzner R G, et al. Cancer Discov 2018 8:1219-26; Schneider D, et al. Sci Transl Med 2021 13:eabc6401). Antigen-binding domains from the FMC63 (anti-CD19) and Leu16 (anti-CD20) antibodies were linked in a tandem configuration and arranged in different orders, with FMC63 furthest from the cell membrane followed by Leu16 (CD19-CD20) or vice versa (CD20-CD19). Both second- (mut06z) generation and third- (BB06z) generation bi-specific CAR T cells were developed (FIG. 11A). All four CAR constructs were significantly expressed on the cell surface at frequencies that ranged from 66% to 91% (FIG. 11A). These four bi-specific constructs also showed different levels of surface CAR expression measured by the median fluorescent intensity within the CAR+population, with 20-19 Tan BB06z showing the highest expression level (online supplemental figure S2A). Prior to antigen exposure, 20-19 Tan BB06z construct showed a higher frequency of CAR T cells with a CM phenotype. To determine the ability of these four bi-specific CAR T cells to individually recognize both target antigens CD19 or CD20 and trigger effector functions an in vitro evaluation of cytotoxicity and cytokine production was performed. All the subsequent experiments were performed with CAR T cells normalized to the construct expressing the lowest CAR frequency by adding UT cells, reaching the same number of CAR T cells and total T cells per condition. All constructs were able to induce cytolysis against CD19-expressing or CD20-expressing cells with different efficiencies, with 20-19 Tan mut06z showing the fastest response and 19-20 Tan mut06z being the least effective. Furthermore, three bi-specific CAR T cells, 19-20 Tan BB06z, 20-19 Tan BB06z, and 20-19 Tan mut06z, showed significant levels of cytokine production when challenged with either CD19 or CD20 while 19-20 Tan mut06z showed no response.

To investigate bi-specific CAR T cell functionality against a tumor cell that naturally expresses both CD19 and CD20 the Raji B cell lymphoma line was used. To further elucidate the impact of CD19 and CD20 a panel of Raji cells expressing varying levels of the CD19 antigen was used as target cells. These include cells with no CD19 expression (Raji-CD19^{KO}), with low levels (Raji-CD19^{Low}), and high levels (Raji-CD19^{High}) of CD19. All four constructs showed effective cytotoxicity by RTCA against Raji-CD19^{KO} compared with no effect of the mono-specific h1928z. Furthermore, all four constructs also showed efficient cytotoxicity against Raji-CD19^{Low} and Raji-CD19^{High} with 20-19 Tan mut06 showing the fastest effector function (FIG. 11B). Additionally, in vitro secretion of IFN-γ, IL-2, TNF-α and IL-6 after 24 hours of co-culture was determined with these three Raji cell lines at a 1:1 ET ratio. Secretion of significant levels of IFN-γ, IL-2, TNF-α, and minimal levels of IL-6 was detected upon antigen engagement with the highest levels observed when both antigens were present. Furthermore, BB06z CAR T cells showed the highest levels of cytokines while mut06z CART cells the lowest (FIG. 11C). The same pattern was observed when using K562 cells artificially engineered to express both human CD19 and CD20. These results suggest that these four bi-specific CAR T cells can recognize both target antigens and elicit a response that varies depending on both the orientation of the antigen-binding domains and the co-stimulatory motifs.

CAR T Cells Combining 4-1BB and mut06 With a CD20/CD19-Oriented Exodomain Show Significant In Vivo Antitumor Activity

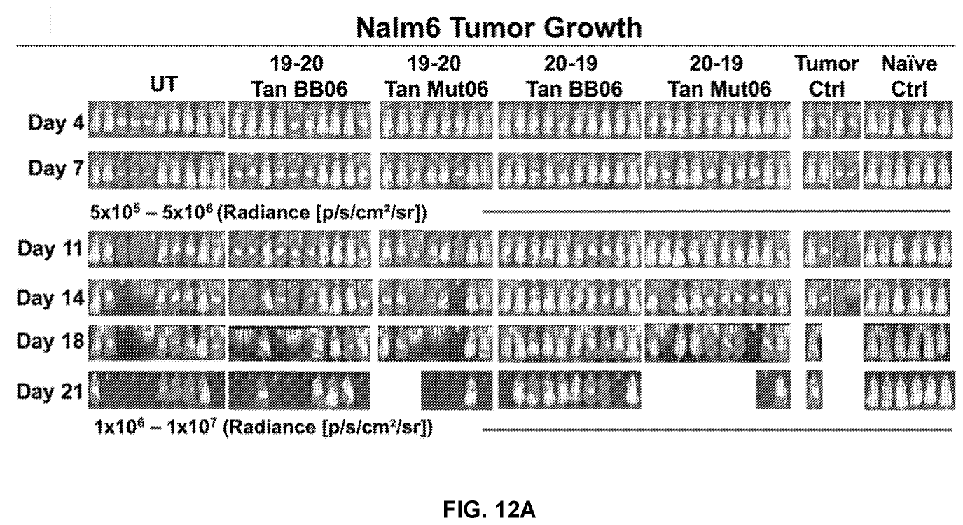

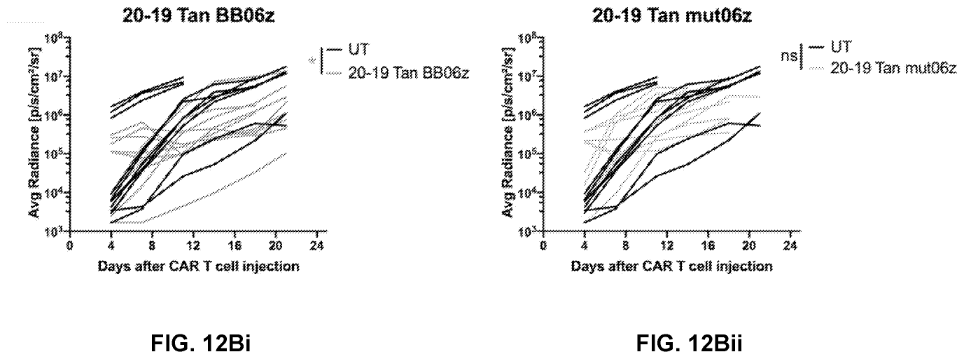

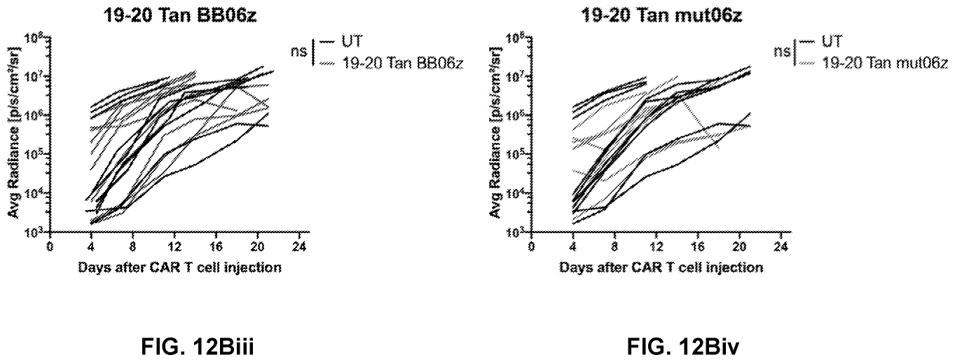

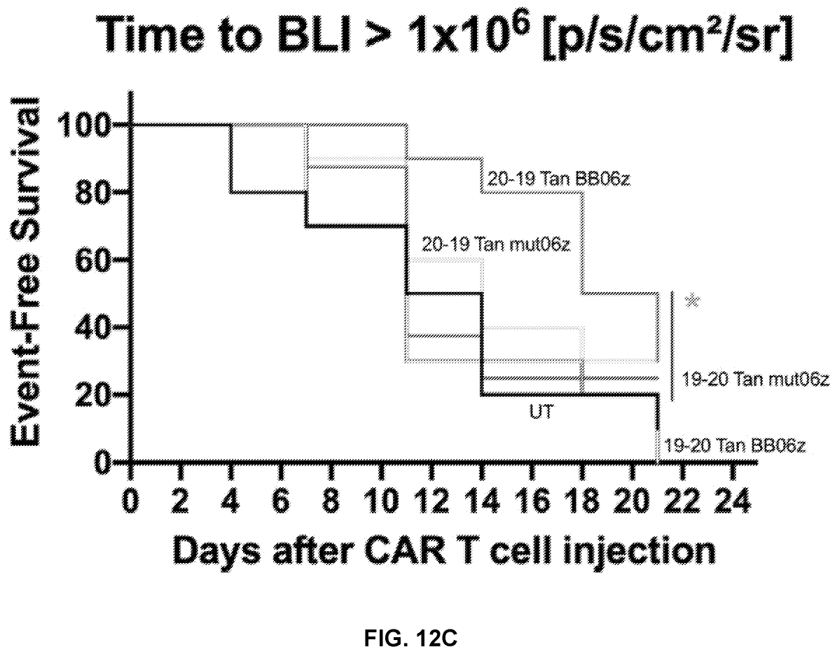

the antitumor function of these four bi-specific CAR T cells were compared in an aggressive xenograft model of acute lymphoblastic leukemia. For this NSG mice were inoculated intravenously with Nalm6-FFluc tumor cells and 1×10⁶ CAR T cells were transferred 7 days later. Tumor growth was monitored by quantitative imaging every week. Both CAR constructs with a CD20-CD19 orientation were able to maintain a low tumor burden at early time points (day 11) compared with CD19-CD20 (FIG. 12A). Furthermore, as the experiment progressed 20-19 BB06z CAR T cells were the only ones that significantly delayed tumor growth compared with UT-treated controls (FIG. 12B). The kinetics of this antitumor effect were also determined by event-free survival, defined as time in days to an average luminescence equal or greater than 1×10⁶ p/s/cm2/sr on these mice. Here it was observed that 20-19 BB06z CAR T cells significantly delayed the progression of the disease while the rest of the groups succumbed sooner (FIG. 12C). These data suggest that the orientation of the anti-CD20 and anti-CD19 scFvs impact the in vivo antitumor efficiency of bi-specific CAR T cells, as 20-19 BB06z shows better response than 19-20 BB06z. Together, the in vitro and in vivo results focused subsequent studies on CD20-CD19 mut06z and BB06z CAR T cells.

CD20/CD19 Bi-Specific CAR T Cells Show Enhanced Antitumor Function Compared With Standard of Care h1928z With Increased Persistence and Lower Exhaustion-Associated Markers

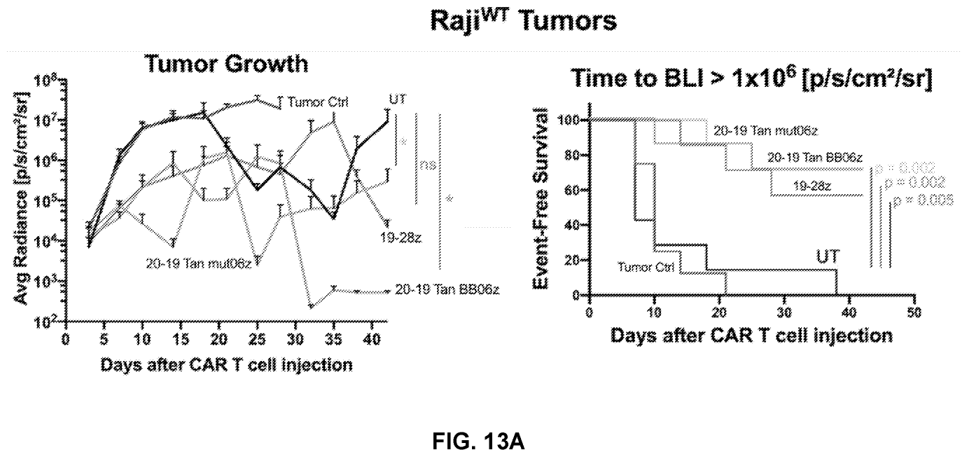

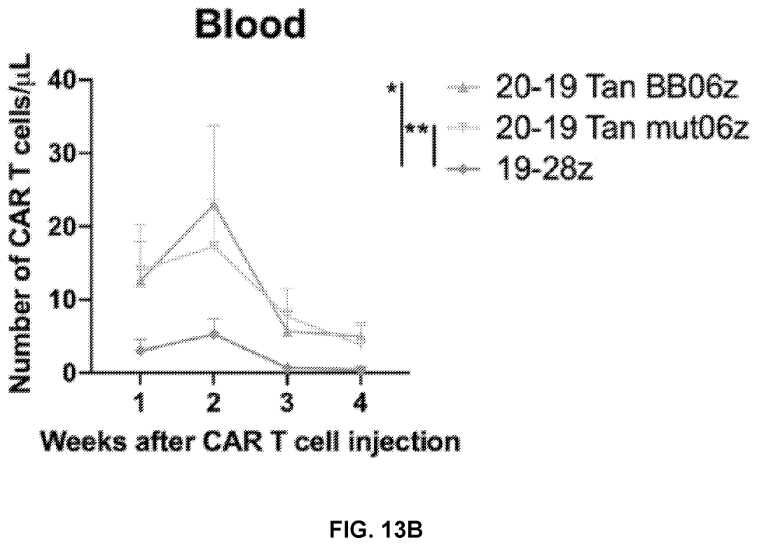

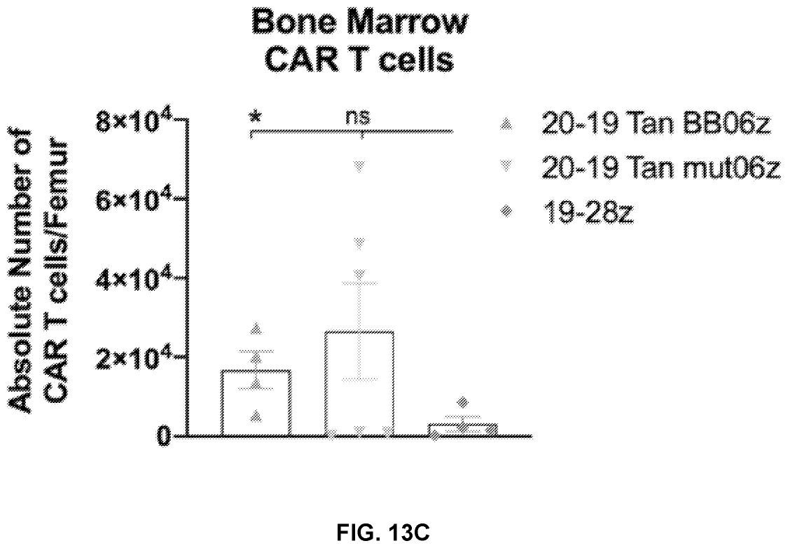

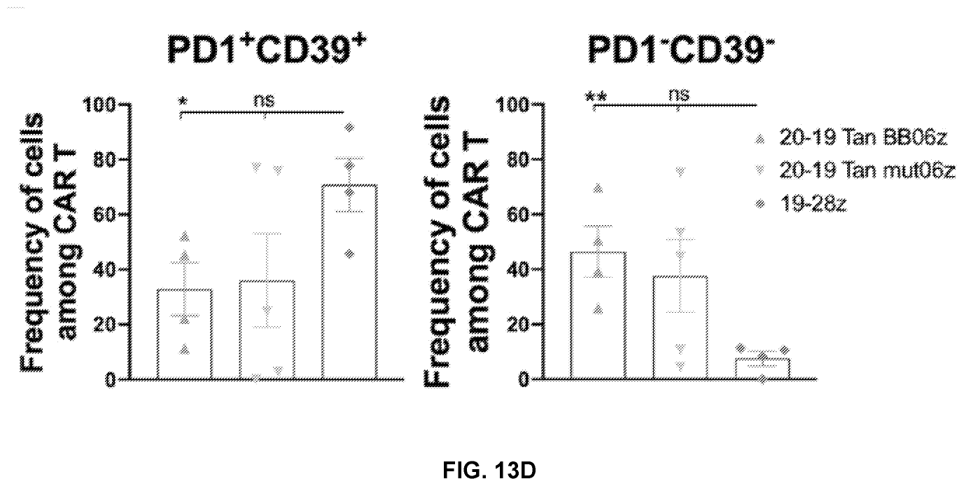

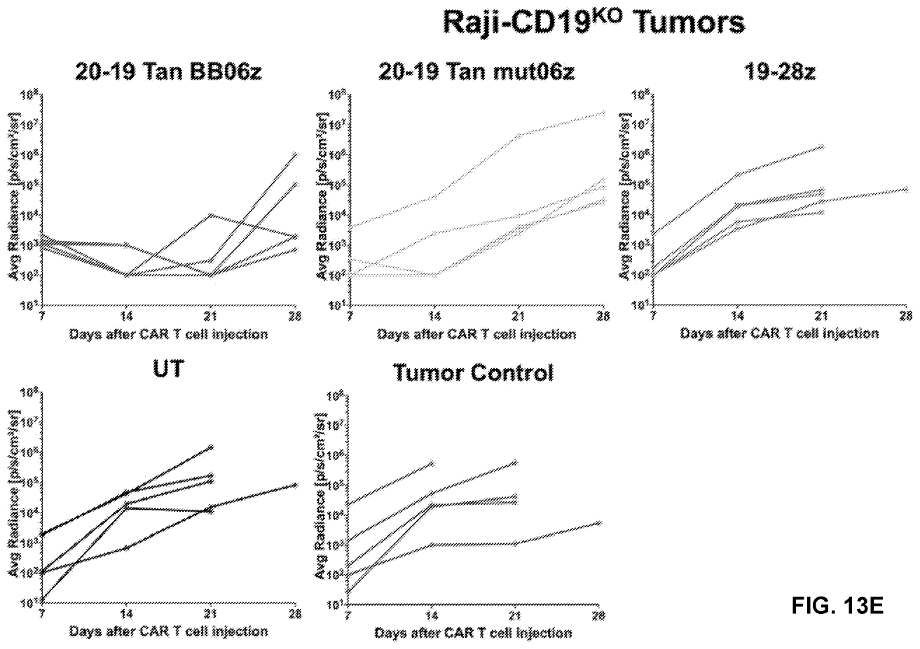

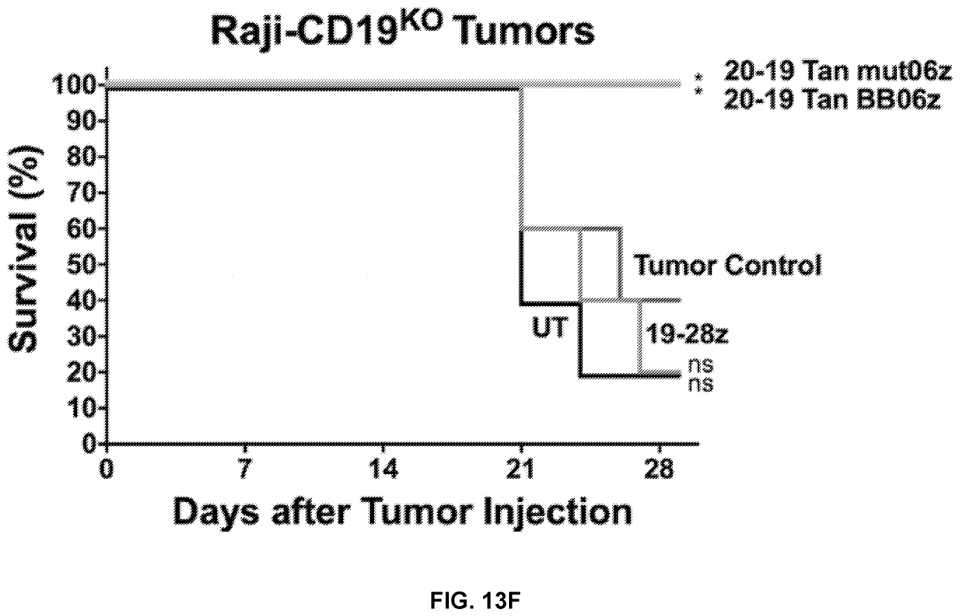

To further investigate the antitumor efficacy of these two bi-specific constructs and evaluate their performance against the standard of care h1928z CAR used in the clinic, a Raji B cell tumor model was used. For this, NSG mice intravenously were inoculated with Raji-FFluc tumor cells and 1×10⁶ CART cells were transferred 5 days later. Tumor growth was monitored by quantitative imaging every week. It was determined that both bi-specific CAR T cells showed significant antitumor activity compared with UT controls (FIG. 13A). By analyzing event-free survival we were able to observe that bi-specific and h1928z CAR T cells showed significant control of tumor progression (FIG. 13A). Furthermore, bi-specific constructs lead to complete tumor regression in some mice while mono-specific CAR T cells controlled tumor growth but with observable tumor burden at the experiment end point (day 42). Moreover, 20-19 Tan BB06z and 20-19 Tan mut06z showed significantly higher number of CAR T cells in peripheral blood compared with h1928z (FIG. 13B). By performing an ex vivo analysis of these mice, it was determined that bi-specific cells showed increased persistence as evidenced by a higher total number of CAR T cells in the bone marrow compared with h1928z (FIG. 13C). Additionally, bi-specific CAR T cells found in the bone marrow showed a lower frequency of PD1⁺CD39⁺ cells, typically associated with an exhausted phenotype. In accordance with this, there was a lower frequency of PD1⁻CD39⁻ in bi-specific CAR T cells (FIG. 13D). All three CAR T cells present in the bone marrow of treated mice showed a predominantly CM phenotype. Furthermore, mono-specific h1928z CAR T cells skewed towards CD8+ cells while 20-19 Tan BB06z showed more CD4+ cells and 20-19 Tan mut06z a more balanced ratio.

To further study the in vivo activity of these cells against only the target antigen CD20 NSG mice were inoculated intravenously with RajiCD19^{KO}-Nanoluc tumor cells that lack the expression of CD19; 1×10⁶ CAR T cells were transferred 5 days later and growth was monitored weekly by bioluminescence. Both bi-specific CAR T cells showed significant antitumor effect while mono-specific h1928z showed no effect, comparable to UT cells. Additionally, 20-19 Tan BB06z CAR T cells showed improved tumor control compared with 20-19 Tan mut06z (FIG. 13E). Both bi-specific CAR T cells were able to significantly prolong the survival of the mice compared with mice receiving UT or h1928z cells (FIG. 13F). Collectively, these data support the hypothesis that CAR T cell activity can be improved by targeting two independent antigens and that the combination of co-stimulatory domains 4-1BB and a mutated form of CD28 shows an improved function in human CAR T cells.

CD20/CD19 8806z CAR T Cells Outperform CD20/CD19 mut06z In Vitro

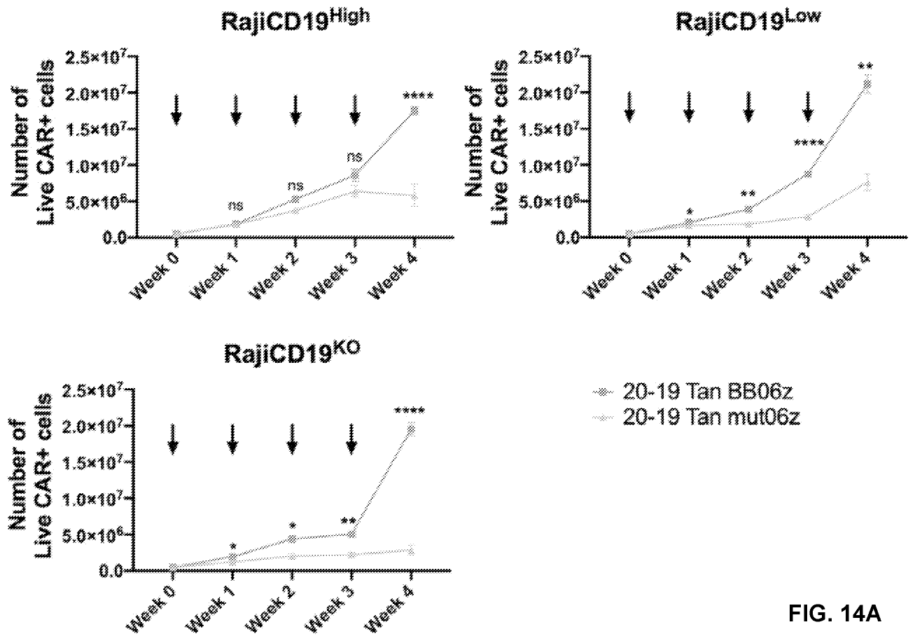

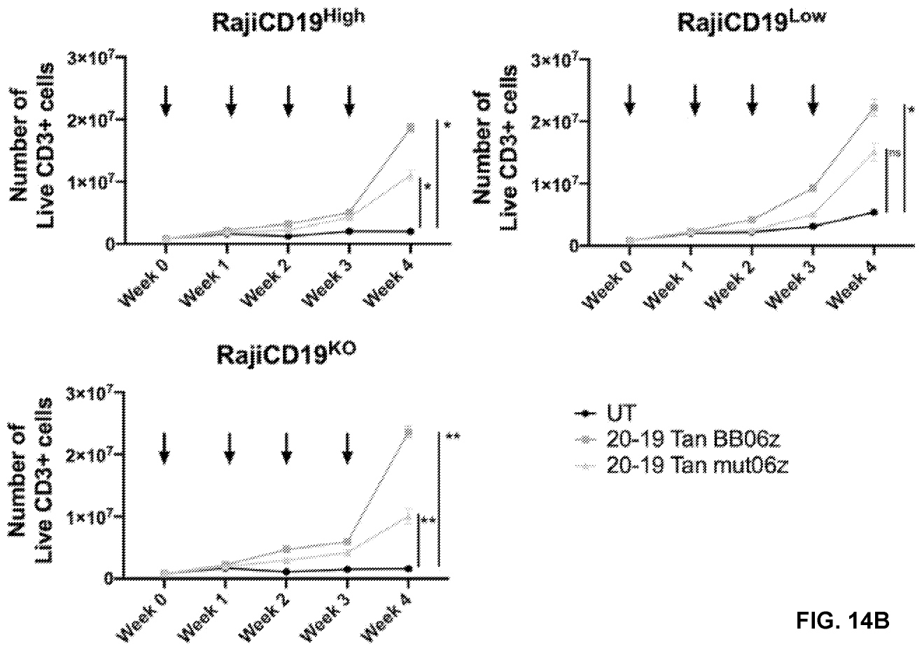

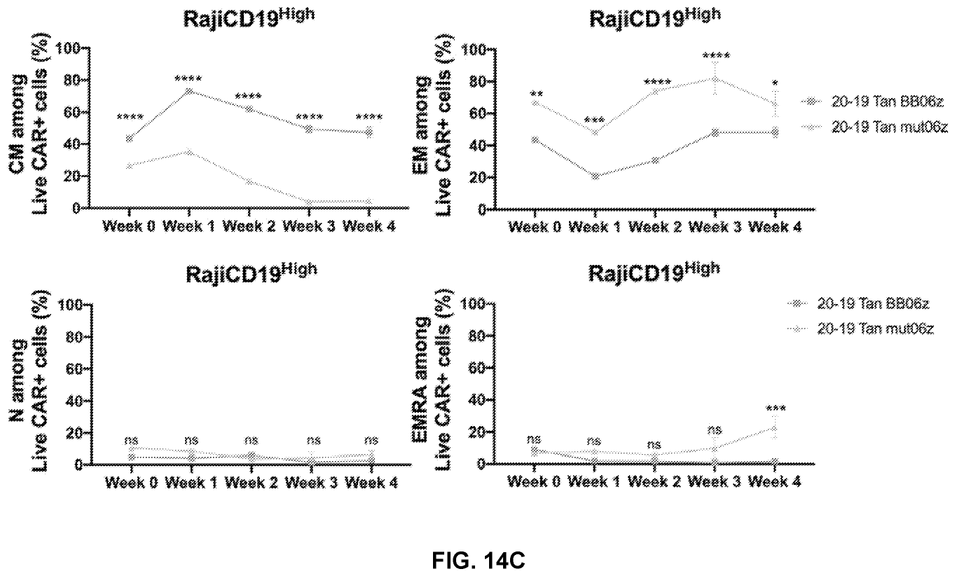

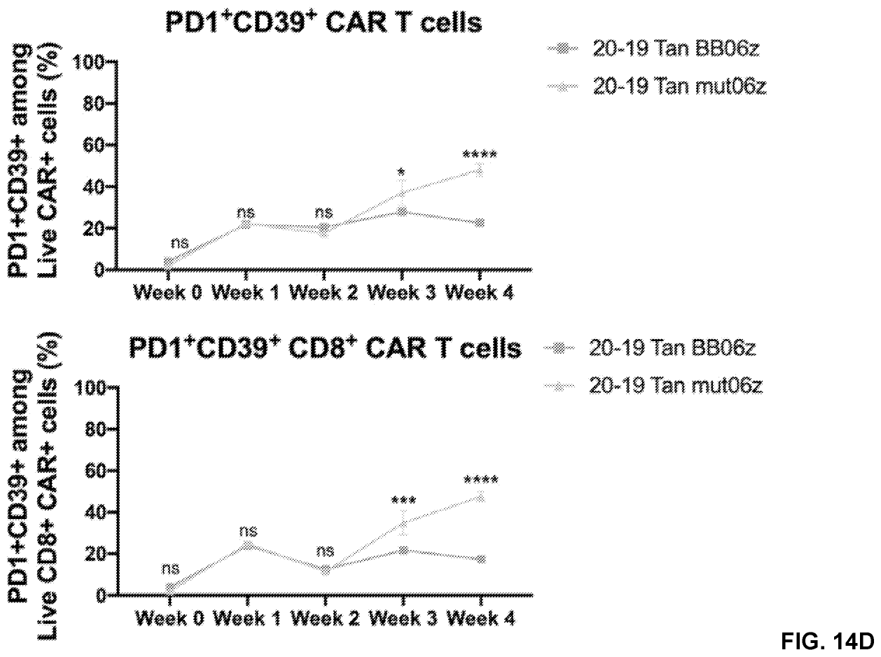

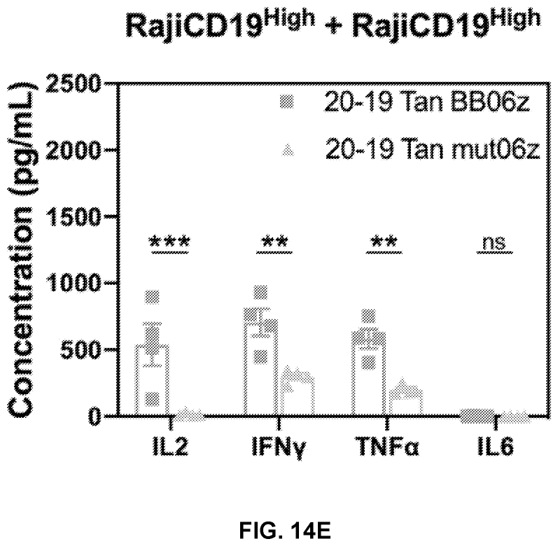

To validate observations in mono-specific CAR T cells regarding polyfunctionality, 20-19 Tan BB06z and 20-19 Tan mut06z CAR T cells were stimulated with RajiWT cells overnight and isolated CD4+ and CD8+ cells before performing the polyfunctional assay. Analyzing four healthy donors it was observed that 20-19 Tan BB06z cells showed higher polyfunctionality and PSI than 20-19 Tan mut06z. To study the response of these CAR T cells to repeated antigenic stimulation an in vitro assay was performed by co-culturing the same initial number of CAR T cells with either Raji-CD19^{KO}, Raji-CD19^{Low}, or Raji-CD19^{High} at a 1:1 E:T ratio. Cells were then counted and analyzed by flow cytometry every 7 days when fresh target cells were added to the co-culture for a total of 4 weeks. 20-19 BB06z CAR T showed significantly superior antigen-dependent expansion than 20-19 mut06z against the three different target cell lines (FIG. 14A). The total number of T cells also increased significantly more in 20-19 BB06z while UT controls did not expand in the experiment (FIG. 14B). Additionally, the T cell differentiation state was evaluated along the re-stimulation by investigating CCR7 and CD45RA expression. 20-19 BB06z CAR T showed a significantly higher CM phenotype frequency while 20-19 Tan mut06z skewed towards effector memory and terminally differentiated effector memory cells re-expressing CD45RA (EMRA) after repeated stimulation with RajiCD19High target cells (FIG. 14C). The same pattern was observed against Raji-CD19^{KO} and Raji-CD19^{Low}.

The expression of the exhaustion-associated markers PD1 and CD39 was also examined on these cells and observed that 20-19 Tan BB06z CAR T cells were more resistant to acquiring this phenotype (FIG. 14D). On repeated stimulation with RajiCD19^{High} target cells, 20-19 Tan BB06z CAR T cells showed a significantly lower frequency of PD1⁺CD39⁺ cells compared with 20-19 Tan mut06z from week 3 onwards. This lower frequency of PD1⁺CD39⁺20-19 Tan BB06z cells was observed on total CAR+ cells and CD8⁺CAR T cells (FIG. 14D). The same results were observed against Raji-CD19^{KO} and Raji-CD19^{Low}. The ratio of CD4/CD8 was also studied within the CAR+ population. 20-19 Tan BB06z cells showed a ratio close to 1 while 20-19 Tan mut06z cells skewed towards CD8 when challenged with all three Raji cell lines. Lastly, after 4 weeks of re-stimulation CAR T cells were counted, washed and re-challenged with RajiCD19^{High} target cells for 24 hours to evaluate cytokine production. To control for the enhanced expansion of 20-19 Tan BB06z the same number of CAR+ T cells was used for each group. 20-19 Tan BB06z secreted significantly higher levels of IL-2, IFN-γ, and TNF-α than 20-19 Tan mut06z while neither secreted significant levels of IL-6 (FIG. 14E). The same was observed for CAR T cells that were co-cultured for 4 weeks with Raji-CD19^{Low} and then re-challenged with RajiCD19^{High}. These data collectively suggest that 20-19 Tan BB06z CAR T cells can secrete a wider array of cytokines (polyfunctionality) and can expand better after repeated antigenic stimulation than 20-19 Tan mut06z while maintaining a favorable memory-like phenotype. These third-generation CAR T cells can also resist exhaustion and show an enhanced ability to produce cytokines when re-challenged with antigen.

### Discussion

Previous research shows the design of a CAR can impact its signaling and function (Roselli E, et al. J Clin Invest 2021 131). Many of these studies focus on how a single co-stimulatory domain, typically 4-1BB or CD28, can determine CAR efficacy. There are conflicting reports on whether combining CD28 and 4-1BB co-stimulation increases efficacy. Several studies in mouse models (Zhong X-S, et al. Mol Ther 2010 18:413-20; Wang J, et al. Hum Gene Ther 2007 18:712-25) and clinical studies (Till B G, et al. Blood 2012 119:3940-50) show no enhancement of tumor killing or patient survival compared with second-generation CARs, in the context of CD19 and B cell malignancies. The concept of finding the perfect balance of signal quantity and quality downstream CAR activation has been extensively examined in recent years. The modification of one amino acid in the CD28 co-stimulatory domain (CD28-YMFM) was able to render CAR T cells resistant to exhaustion and reduced T cell differentiation (Guedan S, et al. J Clin Invest 2020 130:3087-97). Another study showed that by strategically mutating immunoreceptor tyrosine-based activation motifs in CD3ζ of a CD28-based CAR the therapeutic effect was enhanced, with CAR T cells showing improved persistence, a less differentiated phenotype and lower exhaustion (Feucht J, et al. Nat Med 2019 25:82-8). In this work it was shown that by designing a CAR that includes 4-1BB and our mutant CD28 (mut06) we could enhance cell function by optimizing signaling and have the most advantageous aspects of both co-stimulatory domains.

It has been previously shown that including the CD28 co-stimulatory domain can increase CAR expression (Zhang T, et al. J Immunol 2012 189:2290-9). A co-stimulatory domain composed of 4-1BB followed by mut06 resulted in higher surface CAR expression in both mono-specific and bi-specific CAR T cells. This increase in CAR expression may allow better clinical outcomes by reducing the number of initial T cells to produce a viable product. It was recently reported that tisagenlecleucel (4-1BB-based) showed reduced ability to perform against low-antigen density targets compared with axicabtagene ciloleucel (CD28-based) (Majzner R G, et al. Cancer Discov 2020 10:702-23); higher levels of CAR expression on 4-1BB/mut06 cells could increase this threshold and improve the efficacy of 4-1BB-based CARs. Elevated CAR expression can also amplify tonic signaling resulting in poor persistence (Gomes-Silva D, et al. Cell Rep 2017 21:17-26; Guedan S, et al. JCI Insight 20183; Frigault M J, et al. Cancer Immunol Res 2015 3:356-67). However, while CAR T cells combining 4-1BB and mut06 (mono-specific and bi-specific) have the highest CAR expression, they show the greatest expansion after 4 weeks of repeated antigen stimulation. Several studies support the concept that T cells with a less differentiated state and memory features are associated with an improved antitumor effect and persistence in adoptive cell therapy (ACT) (Feucht J, et al. Nat Med 2019 25:82-8; Fraietta J A, et al. Nat Med 2018 24:563-71). Previous work shows that 4-1BB-based CAR T cells show an increased CM phenotype (Kawalekar O U, et al. Immunity 2016 44:380-90). Here it was shown that constructs combining 4-1BB with mut06 maintain a memory-like phenotype after repeated antigenic stimulation that is superior to mut06 or 4-1BB alone.

It was observed that h1906z CAR T cells secrete lower levels of cytokines compared with h1928z which is consistent with mouse data showing mut06 reduces cytokine secretion compared with unmutated CD28 co-stimulation (Boucher J C, et al. Cancer Immunol Res 2021 9:62-74). It was also shown that mut06 CAR T cells have lower polyfunctionality, which can be increased by combining it with 4-1BB. This ability to produce a wider range of cytokines at a single-cell level is associated with an improved clinical response (Rossi J, et al. Blood 2018 132:804-140. While cytokine secretion of h19BB06z CAR T cells was increased compared with mut06, their short-term cytotoxicity was comparable to 4-1BB CARs. However, on repeated antigen stimulation, h19BB06z CAR was able to maintain the greatest cytotoxic ability and cytokine secretion. Different strategies have been studied in order to exploit the beneficial aspects of the IL-2 axis on ACT. Some include mutated versions of IL-2 with higher affinity for IL-2Rβ. (Levin A M, et al. Nature 2012 484:529-33), or the use of an engineered IL-2 cytokine conjugated to polyethylene glycol that enhances ACT (Parisi G, et al. Nat Commun 2020 11:660). Here it was shown that by combining 4-1BB and mut06, both in mono-specific or bi-specific CAR T cells, a cell can be obtained able to secrete high levels of endogenous IL-2 after repeated stimulation that could act paracrinally and/or autocrinally improving the efficacy of these transferred cells.

A recent report demonstrated that 4-1BB CART cell cytotoxicity can be enhanced by recruiting LCK to the CAR complex (Sun C, et al. Cancer Cell 2020 37:216-25) suggesting that a third-generation CAR which increased LCK would be more effective. Co-stimulation with both mut06 and 4-1BB increased pLCK in CAR T cells. A portion of this pLCK was associated with the CAR and that this depended on the order of the co-stimulatory domains.

In vitro data suggest that h1906BBz and h19BB06z CAR T cells have similar functional abilities after initial antigen exposure. However, after multiple challenges with antigen, h19BB06z CAR T cells showed the ability to maintain a memory-like phenotype and expanded significantly more than the other constructs. These observations support a recent study where 4-1BB activation of the NF-κB pathway enhances the in vitro survival of CAR T cells with lower expression of apoptotic factors compared with CD28-based co-stimulation (Philipson B I, et al. Sci Signal 2020 13:eaay8248). When these CARs were examined in vivo h19BB06z had enhanced tumor killing compared with h1906BBz. All this suggests that the positioning of the co-stimulatory domains is critical for CAR function. Previous work shows that having CD28 proximal to the cell membrane followed by 4-1BB increases B-cell acute lymphoblastic leukemia tumor regression in xenograft models (Zhao Z, et al. Cancer Cell 2015 28:415-28). Likewise, data show that having the 4-1BB co-stimulatory domain proximal to the cell membrane can enhance CAR T cell function. This may be due to differences in signaling cascades triggered by 4-1BB and mut06 where this orientation appears to be more effective.

Observations were validated using a different extracellular domain able to recognize both CD19 and CD20. These bi-specific CAR T cells independently recognized both target antigens and elicited an efficient effector function, which may prove beneficial in scenarios where CD19 is lost or downregulated (Majzner R G, et al. Cancer Discov 2018 8:1219-26; Zah E, et al. Cancer Immunol Res 2016 4:498-508). Moreover, both antiCD20/CD19 BB06z and mut06z cells showed efficient in vivo antitumor activity compared with standard anti-CD19-28z CAR T cells. These cells also showed a more favorable memory phenotype associated with increased antitumor potency and persistence (Fraietta J A, et al. Nat Med 2018 24:563-71; Deng Q, et al. Nat Med 2020 26:1878-87) with less exhaustion-associated markers than second-generation 20-19 mut06z cells.

In a recent report, tandem anti-CD19/CD20 CART cells with 4-1BB as co-stimulation were evaluated in a phase-1 study for relapsed, refractory B cell malignancies. They demonstrate therapeutic safety and efficacy, however these cells skew towards an effector-like phenotype prior to infusion (Shah N N, et al. Nat Med 2020 26:1569-75). These observations suggest a possible clinical application of newly developed third-generation bi-specific CAR T cells which showed an improved memory-like phenotype that may even be more beneficial by enhancing the efficacy of this approach. Together this work demonstrates that the activity of CAR T cells can be enhanced by fine-tuning the signaling domains, specifically, by combining 4-1BB and an optimized form of CD28 co-stimulation.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric antigen receptor (CAR) polypeptide, wherein the CAR polypeptide is defined by the formula:
SP-CD19VH-CD20VH-LKR-CD20VL-CD19VL-CD8-41BB-CD28-CD3z;
SP-CD20VH-CD19VH-LKR-CD19VL-CD20VL-CD8-41BB-CD28-CD3z;
SP-CD19VL-CD20VL-LKR-CD20VH-CD19VH-CD8-41BB-CD28-CD3z;
SP-CD20VL-CD19VL-LKR-CD19VH-CD20VH-CD8-41BB-CD28-CD3z;
SP-CD19VH-CD20VH-LKR-CD20VL-CD19VL-CD8-CD28-CD3z;
SP-CD20VH-CD19VH-LKR-CD19VL-CD20VL-CD8-CD28-CD3z;
SP-CD19VL-CD20VL-LKR-CD20VH-CD19VH-CD8-CD28-CD3z;
SP-CD20VL-CD19VL-LKR-CD19VH-CD20VH-CD8-CD28-CD3z;
SP-CD19VH-CD19VL-CD20VH-CD20VL-CD8-41BB-CD28-CD3z;
SP-CD19VL-CD19VH-CD20VL-CD20VH-CD8-41BB-CD28-CD3z;
SP-CD19VH-CD19VL-CD20VL-CD20VH-CD8-41BB-CD28-CD3z;
SP-CD19VL-CD19VH-CD20VH-CD20VL-CD8-41BB-CD28-CD3z;
SP-CD20VH-CD20VL-CD19VH-CD19VL-CD8-41BB-CD28-CD3z;
SP-CD20VL-CD20VH-CD19VL-CD19VH-CD8-41BB-CD28-CD3z;
SP-CD20VH-CD20VL-CD19VL-CD19VH-CD8-41BB-CD28-CD3z;
SP-CD20VL-CD20VH-CD19VH-CD19VL-CD8-41BB-CD28-CD3z;
SP-CD19VH-CD19VL-CD20VH-CD20VL-CD8-CD28-CD3z;
SP-CD19VL-CD19VH-CD20VL-CD20VH-CD8-CD28-CD3z;
SP-CD19VH-CD19VL-CD20VL-CD20VH-CD8-CD28-CD3z;
SP-CD19VL-CD19VH-CD20VH-CD20VL-CD8-CD28-CD3z;
SP-CD20VH-CD20VL-CD19VH-CD19VL-CD8-CD28-CD3z;
SP-CD20VL-CD20VH-CD19VL-CD19VH-CD8-CD28-CD3z;
SP-CD20VH-CD20VL-CD19VL-CD19VH-CD8-CD28-CD3z; or
SP-CD20VL-CD20VH-CD19VH-CD19VL-CD8-CD28-CD3z;
wherein “SP” represents an optional signal peptide,
wherein “CD19VH” represents an anti-CD19 V_{H} domain comprising the amino acid sequence
(SEQ ID NO: 1)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT
or
(SEQ ID NO: 2)
DIELTQSPKFMSTSVGDRVSVTCKASQNVGTNVAWYQQKPGQSPKPLIYS
ATYRNSGVPDRFTGSGSGTDFTLTITNVQSKDLADYFCQQYNRYPYTSGG
GTKLEIK,
wherein “CD19VL” represents an anti-CD19 V_{L} domain comprising the amino acid sequence
(SEQ ID NO: 3)
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSAAA
or
(SEQ ID NO: 4)
EVKLQQSGAELVRPGSSVKISCKASGYAFSSYWMNWVKQRPGQGLEWIGQ
IYPGDGDTNYNGKFKGQATLTADKSSSTAYMQLSGLTSEDSAVYFCARKT
ISSVVDFYFDYWGQGTTVTVSS,
wherein “CD20VH” represents an anti-CD20 V_{H} domain comprising the amino acid sequence
(SEQ ID NO: 5)
EVQLQQSGAELVKPGASVKMSCKASGYTFTSYNMHWVKQTPGQGLEWIGA
IYPGNGDTSYNQKFKGKATLTADKSSSTAYMQLSSLTSEDSADYYCARSN
YYGSSYWFFDVWGAGTTVTVSS,
wherein “CD20VL” represents an anti-CD20 V_{L} domain comprising the amino acid sequence
(SEQ ID NO: 6)
DIVLTQSPAILSASPGEKVTMTCRASSSVNYMDWYQKKPGSSPKPWIYAT
SNLASGVPARFSGSGSGTSYSLTISRVEAEDAATYYCQQWSFNPPTFGGG
TKLEIK,
wherein “LKR” represents a loop linker domain,
wherein “CD8” represents a CD8 hinge domain,
wherein “41BB” represents a 41BB domain,
wherein “CD28” represents a CD28 co-stimulatory signaling region,
wherein “CD3z” represents a CD3 zeta (CD3ζ) region, and
wherein “—” represents a peptide bond or linker.

2. The CAR polypeptide of claim 1, wherein the CD3 zeta region comprises the amino acid sequence
(SEQ ID NO: 7)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

3. The CAR polypeptide of claim 1, wherein the 41BB region comprises the amino acid sequence
(SEQ ID NO: 8)
RGRKKLLYIFKQPFMRPVQTTQEEDGCSCRFPEEEEGGCEL.

4. The CAR polypeptide of claim 1, wherein the CD8 hinge domain comprises the amino acid sequence
(SEQ ID NO: 9)
TTTPAPRPPTPAPTIASQPLSLRPEACRPAAGGAVHTRGLDFACDIYIWA
PLAGTCGVLLLSLVITLYC.

5. The CAR polypeptide of claim 1, wherein the loop linker domain comprises the amino acid sequence GSTSGSGKPGSGEGSTKG (SEQ ID NO:10).

6. The CAR polypeptide of claim 1, wherein the CD28 co-stimulatory signaling region comprises a cytoplasmic domain of CD28 having a null mutation in the tyrosine amino acid of the YMNM (SEQ ID NO:11) subdomain, and wherein the co-stimulatory signaling region comprises a cytoplasmic domain of CD28 having a null mutation in the proline amino acids of the PRRP (SEQ ID NO:12) subdomain.

7. The CAR polypeptide of claim 6, wherein the CD28 co-stimulatory signaling region comprises a cytoplasmic domain of CD28 having a wildtype PYAP (SEQ ID NO:13) subdomain.

8. The CAR polypeptide of claim 6, wherein the CD28 co-stimulatory signaling region comprises the amino acid sequence RSKRSRLLHSDX₁MNMTX₂RRX₃GPTRKHYQPYAPPRDFAAYRS, wherein X₁ is not Y, and wherein X₂ and X₃ are not P (SEQ ID NO:14), or an amino acid sequence having at least 95% sequence identity to SEQ ID NO:14.

9. The CAR polypeptide of claim 8, wherein the X₁, X₂, and X₃ are conservative substitutions.

10. The CAR polypeptide of claim 6, wherein the CD28 co-stimulatory signaling region comprises the amino acid sequence
(SEQ ID NO: 15)
RSKRSRLLHSDFMNMTARRAGPTRKHYQPYAPPRDFAAYRS.

11. A bi-specific CAR T cell comprising an immune effector cell engineered to express at least one of the chimeric antigen receptor polypeptide(s) of claim 1.

12. The bi-specific CART cell of claim 11, wherein the immune effector cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

13. A method of providing an anti-cancer immunity in a subject with a myeloid or B cell malignancy, the method comprising administering to the subject an effective amount of the CAR T cell of claim 11, thereby providing an anti-tumor immunity in the mammal.

14. The method of claim 13, further comprising administering to the subject a checkpoint inhibitor.

15. The method of claim 14 wherein the checkpoint inhibitor comprises an anti-PD-1 antibody, anti-PD-L1 antibody, anti-CTLA-4 antibody, or a combination thereof.

16. The method of claim 13, wherein the myeloid or B cell malignancy comprises Acute Myeloid Leukemia (AML), blastic plasmocytoid dendritic cell neoplasm, hairy cell leukemia, and Acute Lymphoblastic Leukemia.

17. The method of claim 13, wherein CAR T cells administered to the mammal are autologous to the subject.

18. The method of claim 13, wherein CAR T cells administered to the mammal are allogeneic to the subject.


SEQUENCE LISTING (17 sequences)

SEQ ID NO: 1 - protein, 107 aa, Artificial Sequence
  Synthetic Construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 2 - protein, 107 aa, Artificial Sequence
  Synthetic Construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIK

SEQ ID NO: 3 - protein, 123 aa, Artificial Sequence
  Synthetic Construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAA

SEQ ID NO: 4 - protein, 122 aa, Artificial Sequence
  Synthetic Construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 5 - protein, 122 aa, Artificial Sequence
  Synthetic Construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 6 - protein, 106 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 7 - protein, 112 aa, Artificial Sequence
  Synthetic Construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 41 aa, Artificial Sequence
  Synthetic Construct
RGRKKLLYIFKQPFMRPVQTTQEEDGCSCRFPEEEEGGCEL

SEQ ID NO: 9 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 10 - protein, 18 aa, Artificial Sequence
  Synthetic Construct
GSTSGSGKPGSGEGSTKG

SEQ ID NO: 11 - protein, 4 aa, Artificial Sequence
  Synthetic Construct
YMNM

SEQ ID NO: 12 - protein, 4 aa, Artificial Sequence
  Synthetic Construct
PRRP

SEQ ID NO: 13 - protein, 4 aa, Artificial Sequence
  Synthetic Construct
PYAP

SEQ ID NO: 14 - protein, 41 aa, Artificial Sequence
  Synthetic Construct
  MISC_FEATURE (12)..(12): Any amino acid other than Tyr
  MISC_FEATURE (17)..(17): Any amino acid other than Pro
  MISC_FEATURE (20)..(20): Any amino acid other than Pro
RSKRSRLLHSDXMNMTXRRXGPTRKHYQPYAPPRDFAAYRS

SEQ ID NO: 15 - protein, 41 aa, Artificial Sequence
  Synthetic Construct
RSKRSRLLHSDFMNMTARRAGPTRKHYQPYAPPRDFAAYRS

SEQ ID NO: 16 - protein, 4 aa, Artificial Sequence
  Synthetic Construct
FMNM

SEQ ID NO: 17 - protein, 4 aa, Artificial Sequence
  Synthetic Construct
ARRA